(19) United States
(12) Patent Application Publication
(10) Pub. No.: US 2011/0263594 A1
(43) Pub. Date: Oct. 27, 2011

(54) NOVEL TRIAZOLO(4,3-A)PYRIDINE DERIVATIVES, PROCESS FOR THE PREPARATION THEREOF, USE THEREOF AS MEDICAMENTS, PHARMACEUTICAL COMPOSITIONS AND NOVEL USE, IN PARTICULAR AS MET INHIBITORS

(75) Inventors: Eric Bacque, Paris (FR); Dominique Damour, Paris (FR); Conception Nemecek, Paris (FR); Patrick Nemecek, Paris (FR); Sylvie Wentzler, Paris (FR)

(73) Assignee: SANOFI-AVENTIS, Paris (FR)

(21) Appl. No.: 13/054,663

(22) PCT Filed: Jul. 16, 2009
(86) PCT No.: PCT/FR2009/051406

§ 371 Date: Jul. 11, 2011

(30) Foreign Application Priority Data
Jul. 18, 2008 (FR) 0804084
Jan. 21, 2009 (FR) 0900245

(51) Int. Cl. A61K 31/437; C07D 413/14; A61K 31/5377; A61P 9/00; A61P 7/04; A61P 3/00; A61P 37/00; A61P 11/06; A61P 7/02; A61P 25/00; A61P 9/10; A61P 17/06; A61P 29/00; A61P 3/10; A61P 21/00; A61P 35/00; A61P 35/04; C07D 471/04

(52) U.S. Cl. 514/233.2; 546/119; 544/127; 514/303

(57) ABSTRACT

The invention relates to the novel products of formula (I): in which: Ra represents H, Hal, aryl or heteroaryl, which is optionally substituted; Rb represents H, Rc, —COORc-CO-Rc or —CO—NRcRd; where Rc represents alkyl, cycloalkyl, heterocycloalkyl, aryl and heteroaryl, all optionally substituted; Rd represents H, alk or cycloalkyl; these products being in all the isomer forms and the salts, as medicaments, in particular as MET inhibitors.

The present invention relates to novel triazolo[4,3-a]pyridine derivatives, to the process for the preparation thereof, to the novel intermediates obtained, to the use thereof as medicaments, to the pharmaceutical compositions containing them and to the novel use of such triazolo[4,3-a]pyridine derivatives.

The present invention relates more particularly to novel triazolo[4,3-a]pyridine derivatives having an anticancer activity, via the modulation of the activity of proteins, in particular of kinases.

To date, most of the commercially available compounds used in chemotherapy are cytotoxic agents which pose considerable problems in terms of side effects and tolerance by patients. These effects could be limited if the medicaments used act selectively on cancer cells, to the exclusion of healthy cells. One of the solutions for limiting the adverse effects of a chemotherapy may thus consist in using medicaments that act on metabolic pathways or constituent elements of these pathways, predominantly expressed in cancer cells, and which would be sparingly expressed or not expressed in healthy cells. The protein kinases are a family of enzymes that catalyse the phosphorylation of hydroxyl groups of specific residues of proteins, such as tyrosine, serine or threonine residues. Such phosphorylations can largely modify the function of proteins: thus, protein kinases play an important role in the regulation of a large variety of cell processes, including in particular metabolism, cell proliferation, cell adhesion and motility, cell differentiation or cell survival, certain protein kinases playing a central role in the initiation, development and accomplishment of cell cycle events.

Among the various cellular functions in which the activity of a protein kinase is involved, certain processes represent attractive targets for treating certain diseases. As an example, mention may in particular be made of angiogenesis and the control of the cell cycle and also that of cell proliferation, in which protein kinases can play an essential role. These processes are in particular essential for the growth of solid tumours and also for other diseases: in particular, molecules that inhibit such kinases are capable of limiting unwanted cell proliferations such as those observed in cancers, and may play a part in preventing, regulating or treating neurodegenerative diseases such as Alzheimer's disease or neuronal apoptosis.

A subject of the present invention is novel derivatives with inhibitory effects on protein kinases. The products according to the present invention may thus in particular be used for preventing or treating diseases that may be modulated by inhibition of protein kinases.

The products according to the invention in particular show anticancer activity, via the modulation of the activity of kinases. Among the kinases for which a modulation of the activity is sought, MET and also mutants of the MET protein are preferred.

The present invention also relates to the use of said derivatives for the preparation of a medicament for use in human therapy.

Thus, one of the objects of the present invention is to provide compositions that have an anticancer activity, by acting in particular on kinases. Among the kinases for which a modulation of the activity is sought, MET is preferred.

In the pharmacological section hereinafter, it is shown, in biochemical tests and on cell lines, that the products of the present application thus inhibit in particular the autophosphorylation activity of MET and the proliferation of cells whose growth depends on MET or on mutant forms thereof.

MET, or Hepatocyte Growth Factor Receptor, is a receptor with tyrosine kinase activity, expressed in particular by epithelial and endothelial cells. HGF, Hepatocyte Growth Factor, is described as the specific ligand of MET.

HGF is secreted by the mesenchymal cells and activates the MET receptor, which homodimerizes. Consequently, the receptor autophosphorylates on the tyrosines of the catalytic domain Y1230, Y1234 and Y1235.

Stimulation of MET with HGF induces cell proliferation, scattering (or dispersion) and motility, resistance to apoptosis, invasion and angiogenesis.

MET and likewise HGF are found to be overexpressed in many human tumours and a wide variety of cancers. MET is also found to be amplified in gastric tumours and glioblastomas. Many point mutations of the MET gene have also been described in tumours, in particular in the kinase domain, but also in the juxtamembrane domain and the SEMA domain. Overexpression, amplification or mutations cause constitutive activation of the receptor and dysregulation of its functions.

The present invention thus relates in particular to novel inhibitors of the MET protein kinase and of its mutants, that can be used for antiproliferative and antimetastatic treatment, in particular in oncology.

The present invention also relates to novel inhibitors of the MET protein kinase and of its mutants, that can be used for an anti-angiogenic treatment, in particular in oncology.

A subject of the present invention is the products of formula (I):

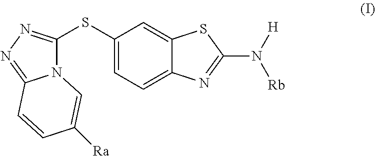

in which:
Ra represents a hydrogen atom; a halogen atom; an aryl radical; or a heteroaryl radical, these aryl and heteroaryl radicals being optionally substituted as indicated hereinafter;
Rb represents a hydrogen atom, an Rc, —COORc or —CO—Rc radical or a —CO—NRcRd radical;
where Rc represents an alkyl, cycloalkyl, heterocycloalkyl, aryl or heteroaryl radical, all these radicals being optionally substituted as indicated hereinafter;
Rd represents a hydrogen atom or an alkyl or cycloalkyl radical;
all the alkyl, cycloalkyl, heterocycloalkyl, aryl and heteroaryl radicals defined above being optionally substituted with one or more radicals chosen from halogen atoms, and hydroxyl, alkoxy, CN, CF₃, —NR1R2, heterocycloalkyl, —COOH, —COOalk, —CONR1R2 and —NR1COR2 radicals;
the alkyl and cycloalkyl radicals also being optionally substituted with an aryl or heteroaryl radical, themselves optionally substituted with one or more radicals chosen from halogen atoms and hydroxyl, alkyl, alkoxy and NR3R4 radicals;
the cycloalkyl, heterocycloalkyl, aryl or heteroaryl radicals also being optionally substituted with an alkyl radical, itself optionally substituted with one or more radicals chosen from halogen atoms and hydroxyl, O-hetero-cycloalkyl, alkyl, alkoxy and NR3R4 radicals;
NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
the cyclic radicals that R1 and R2 or R3 and R4, respectively, can form with the nitrogen atom to which they are attached, being optionally substituted with one or more radicals, which may be identical or different, chosen from halogen atoms, hydroxyl, oxo, alkoxy, NH₂, NHalk and N(alk)₂ radicals, and alkyl, phenyl, CH₂-phenyl and heteroaryl radicals, such that, in the latter radicals, the alkyl, phenyl and heteroaryl radicals are themselves optionally substituted with one or more radicals chosen from halogen atoms and the following radicals: hydroxyl, alkyl and alkoxy containing from 1 to 4 carbon atoms, NH₂, NHalk and N(alk)₂;
all the alkyl (alk) and alkoxy radicals above containing from 1 to 6 carbon atoms,
said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above, in which:

Ra represents a hydrogen atom; a halogen atom; or an aryl or heteroaryl radical, these aryl and heteroaryl radicals being optionally substituted as indicated hereinafter;
Rb represents a hydrogen atom, a —CO—Rc radical or a —CO—NRcRd radical;
where Rc represents an alkyl radical or a cycloalkyl radical, both optionally substituted with one or more radicals chosen from hydroxyl, alkoxy, NR1R2, heterocycloalkyl, aryl and heteroaryl radicals, themselves optionally substituted as indicated hereinafter;
Rd represents a hydrogen atom or an alkyl radical;
all the alkyl, cycloalkyl, heterocycloalkyl, aryl and heteroaryl radicals defined above being optionally substituted with one or more radicals chosen from halogen atoms, and hydroxyl, alkoxy, heterocycloalkyl, —NR1R2, —COOH, —COOalk and —CONR1R2 radicals;
the aryl or heteroaryl radicals also being optionally substituted with an alkyl radical, itself optionally substituted with one or more radicals chosen from halogen atoms and hydroxyl, O-heterocycloalkyl and alkoxy radicals;
NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different, chosen from halogen atoms, hydroxyl and alkoxy radicals, and alkyl, phenyl and CH₂-phenyl radicals, in which the alkyl or phenyl radicals are themselves optionally substituted with one or more radicals, which may be identical or different, chosen from halogen atoms and alkyl, hydroxyl, alkoxy, NH₂, NHalk and N(alk)₂ radicals;
all the alkyl (alk) or alkoxy radicals above containing from 1 to 6 carbon atoms,
said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above or hereinafter, in which:

Ra represents a hydrogen atom; a halogen atom; a phenyl radical optionally substituted as indicated hereinafter; or a pyrazolyl radical optionally substituted with a heterocycloalkyl radical or with an alkyl radical, itself optionally substituted with a hydroxyl radical or with an O-heterocycloalkyl radical;
Rb represents a hydrogen atom, a —CO—Rc radical or a —CO—NRcRd radical;
where Rc represents an alkyl or cycloalkyl radical, both optionally substituted with one or more radicals chosen from the radicals hydroxyl, alkoxy, NR1R2 and phenyl, itself optionally substituted with one or more radicals chosen from halogen atoms, and hydroxyl, alkoxy, alkyl, NH₂, NHalk and N(alk)₂ radicals;
Rd represents a hydrogen atom or an alkyl radical;
NR1R2 is such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, or phenyl radicals, themselves optionally substituted; or R1 and R2 form, with the nitrogen atom to which they are attached, a cyclic radical containing from 4 to 7 ring members and optionally another heteroatom chosen from O, S, N and NH, this radical, including the possible NH that it contains, being optionally substituted;
NR3R4 being such that: either R3 and R4, which may be identical or different, represent a hydrogen atom or an alkyl radical optionally substituted with one or more radicals, which may be identical or different, chosen from hydroxyl or alkoxy radicals; or R3 and R4 form, with the nitrogen atom to which they are attached, a cyclic radical containing from 4 to 7 ring members and optionally another heteroatom chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different, as defined in either one of Claims 1 and 2;
all the alkyl (alk) or alkoxy radicals above containing from 1 to 4 carbon atoms,
said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above or hereinafter, in which:

Ra represents a hydrogen atom; a halogen atom; or a phenyl radical optionally substituted with one or more radicals chosen from halogen atoms and alkyl radicals; or a pyrazolyl radical optionally substituted with a piperidyl radical or with an alkyl radical, itself optionally substituted with a hydroxyl radical or with a tetrahydro-2H-pyran-2-yloxy radical;
Rb represents a hydrogen atom, a —CO-Rc radical or a —CO—NRcRd radical;
where Rc represents an alkyl or cycloalkyl radical optionally substituted with one or more radicals chosen from hydroxyl, alkoxy and NR1R2 radicals;
Rd represents a hydrogen atom;
NR1R2 being such that: either R1 and R2, which may be identical or different, represent a hydrogen atom or an alkyl radical optionally substituted with one or more radicals, which may be identical or different, chosen from hydroxyl, alkoxy, NH₂, NHalk and N(alk)₂ radicals; or R1 and R2 form, with the nitrogen atom to which they are attached, a cyclic radical containing from 4 to 7 ring members and optionally another heteroatom chosen from O, S, N and NH, optionally substituted with an alkyl, phenyl or —CH₂-phenyl radical, the latter radicals being themselves optionally substituted with one or more radicals, which may be identical or different, chosen from halogen atoms and alkyl, hydroxyl, alkoxy, NH₂, NHalk and N(alk)₂ radicals;
all the alkyl (alk) or alkoxy radicals above containing from 1 to 4 carbon atoms,
said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above or hereinafter, in which:

Ra represents a hydrogen atom; an iodine atom; a phenyl radical optionally substituted with one or two radicals chosen from halogen atoms and a methyl radical; or a pyrazolyl radical optionally substituted with a piperidyl radical or with an ethyl radical, itself optionally substituted with a hydroxyl radical or with a tetrahydro-2H-pyran-2-yloxy radical;
Rb represents a hydrogen atom, a CO-Rc radical or a —CO—NRcRd radical;
where Rc represents a cyclopropyl radical or an alkyl radical optionally substituted with an alkoxy or NR1R2 radical;
Rd represents a hydrogen atom;
NR1R2 being such that: either R1 and R2, which may be identical or different, represent a hydrogen atom or an alkyl radical; or R1 and R2 form, with the nitrogen atom to which they are attached, a morpholinyl or piperazinyl radical optionally substituted on the second nitrogen atom with an alkyl radical;
the alkyl and alkoxy radicals above containing from 1 to 4 carbon atoms,
said products of formula (I) being in all the possible racemic, enantiomeric and diastereoisomeric isomer forms, and also the addition salts with inorganic and organic acids or with inorganic and organic bases of said products of formula (I).

The subject of the present invention is thus the products of formula (I):

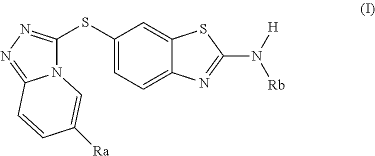

in which:
Ra represents a hydrogen atom; a halogen atom; an aryl radical; or a heteroaryl radical, these aryl and heteroaryl radicals being optionally substituted as indicated hereinafter;
Rb represents a hydrogen atom, an Rc, —COORc or —CO—Rc radical or a —CO—NRcRd radical;
where Rc represents an alkyl, cycloalkyl, heterocycloalkyl, aryl or heteroaryl radical, all these radicals being optionally substituted as indicated hereinafter;
Rd represents a hydrogen atom or an alkyl or cycloalkyl radical;
all the alkyl, cycloalkyl, heterocycloalkyl, aryl or heteroaryl radicals defined above being optionally substituted with one or more radicals chosen from halogen atoms, and hydroxyl, alkoxy, CN, CF₃, —NR1R2, —COOH, —COOalk, —CONR1R2 and —NR1COR2 radicals;
the alkyl and cycloalkyl radicals also being optionally substituted with a heterocycloalkyl, aryl or heteroaryl radical, themselves optionally substituted with one or more radicals chosen from halogen atoms and hydroxyl, alkyl, alkoxy and NR3R4 radicals;
the cycloalkyl, heterocycloalkyl, aryl or heteroaryl radicals also being optionally substituted with an alkyl radical, itself optionally substituted with one or more radicals chosen from halogen atoms and hydroxyl, alkyl, alkoxy and NR3R4 radicals;
NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different, chosen from halogen atoms, hydroxyl, oxo, alkoxy, NH₂, NHalk and N(alk)₂ radicals, and alkyl, phenyl, CH₂-phenyl and heteroaryl radicals, such that, in the latter radicals, the alkyl, phenyl and heteroaryl radicals are themselves optionally substituted with one or more radicals chosen from halogen atoms and the following radicals: hydroxyl, alkyl and alkoxy containing from 1 to 4 carbon atoms, NH₂, NHalk and N(alk)₂;
all the alkyl (alk) and alkoxy radicals above containing from 1 to 6 carbon atoms,
said products of formula (I) being in all the possible racemic, enantiomeric and diastereoisomeric isomer forms, and also the addition salts with inorganic and organic acids or with inorganic and organic bases of said products of formula (I).

In particular, in the products of formula (I),

all the alkyl, cycloalkyl, heterocycloalkyl, aryl and heteroaryl radicals defined above are optionally substituted with one or more radicals chosen from halogen atoms, and hydroxyl, alkoxy, CN, CF₃, —NR1R2, —COOH, —COOalk, —CONR1R2 and —NR1COR2 radicals;
the alkyl radicals also being optionally substituted with an aryl or heteroaryl radicals, themselves optionally substituted with one or more radicals chosen from halogen atoms and hydroxyl, alkyl, alkoxy and NR3R4 radicals;
the cycloalkyl, heterocycloalkyl, aryl or heteroaryl radicals also being optionally substituted with an alkyl radical, itself optionally substituted with one or more radicals chosen from halogen atoms and hydroxyl, alkyl, alkoxy and NR3R4 radicals.

A subject of the present invention is the products of formula (I) as defined above, in which:

Ra represents a hydrogen atom; a halogen atom; or an aryl or heteroaryl radical, these aryl and heteroaryl radicals being optionally substituted as indicated hereinafter;
Rb represents a hydrogen atom, a —CO—Rc radical or a —CO—NRcRd radical;
where Rc represents an alkyl radical or a cycloalkyl radical, both optionally substituted with one or more radicals chosen from hydroxyl, alkoxy, NR1R2, heterocycloalkyl, aryl and heteroaryl radicals, themselves optionally substituted as indicated hereinafter;
Rd represents a hydrogen atom or an alkyl radical;
all the alkyl, cycloalkyl, heterocycloalkyl, aryl and heteroaryl radicals defined above being optionally substituted with one or more radicals chosen from halogen atoms and hydroxyl, alkoxy, —NR1R2, —COOH, —COOalk and —CONR1R2 radicals;
NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different, chosen from halogen atoms, hydroxyl and alkoxy radicals, and alkyl, phenyl and CH₂-phenyl radicals, in which the alkyl or phenyl radicals are themselves optionally substituted with one or more radicals, which may be identical or different, chosen from halogen atoms and alkyl, hydroxyl, alkoxy, NH₂, NHalk and N(alk)₂ radicals;
all the alkyl (alk) or alkoxy radicals above containing from 1 to 6 carbon atoms,
said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above or hereinafter, in which:

Ra represents a hydrogen atom; a halogen atom; or a phenyl radical which is optionally substituted as indicated hereinafter;
Rb represents a hydrogen atom, a —CO—Rc radical or a —CO—NRcRd radical;
where Rc represents an alkyl or cycloalkyl radical, both optionally substituted with one or more radicals chosen from the radicals hydroxyl, alkoxy, NR1R2 and phenyl, itself optionally substituted with one or more radicals chosen from halogen atoms, and hydroxyl, alkoxy, alkyl, NH₂, NHalk and N(alk)₂ radicals;
Rd represents a hydrogen atom or an alkyl radical;
NR1R2 is such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, or phenyl radicals, themselves optionally substituted; or R1 and R2 form, with the nitrogen atom to which they are attached, a cyclic radical containing from 4 to 7 ring members and optionally another heteroatom chosen from O, S, N and NH, this radical, including the possible NH that it contains, being optionally substituted;
NR3R4 being such that: either R3 and R4, which may be identical or different, represent a hydrogen atom or an alkyl radical optionally substituted with one or more radicals, which may be identical or different, chosen from hydroxyl or alkoxy radicals; or R3 and R4 form, with the nitrogen atom to which they are attached, a cyclic radical containing 4 to 7 ring members and optionally another heteroatom chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different, as defined above;
all the alkyl and alkoxy radicals above containing from 1 to 4 carbon atoms;
said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above or hereinafter, in which:

Ra represents a hydrogen atom; a halogen atom; or a phenyl radical optionally substituted with a halogen atom;
Rb represents a hydrogen atom, a —CO—Rc radical or a —CO—NRcRd radical;
where Rc represents an alkyl or cycloalkyl radical optionally substituted with one or more radicals chosen from hydroxyl, alkoxy and NR1R2 radicals;
Rd represents a hydrogen atom;
NR1R2 being such that: either R1 and R2, which may be identical or different, represent a hydrogen atom or an alkyl radical optionally substituted with one or more radicals, which may be identical or different, chosen from hydroxyl, alkoxy, NH₂, NHalk and N(alk)₂ radicals; or R1 and R2 form, with the nitrogen atom to which they are attached, a cyclic radical containing from 4 to 7 ring members and optionally another heteroatom chosen from O, S, N and NH, optionally substituted with an alkyl, phenyl or —CH₂-phenyl radical, the latter radicals being themselves optionally substituted with one or more radicals, which may be identical or different, chosen from halogen atoms and alkyl, hydroxyl, alkoxy, NH₂, NHalk and N(alk)₂ radicals;
all the alkyl (alk) or alkoxy radicals above containing from 1 to 4 carbon atoms,
said products of formula (I) being in all the possible racemic, enantiomeric and diastereoisomeric isomer forms, and also the addition salts with inorganic and organic acids or with inorganic and organic bases of said products of formula (I).

In the products of formula (I) and in the text hereinbelow:
the term “alkyl (or alk) radical” denotes linear and, where appropriate, branched methyl, ethyl, propyl, isopropyl, butyl, isobutyl, sec-butyl, tert-butyl, pentyl, isopentyl, hexyl, isohexyl and also heptyl, octyl, nonyl and decyl radicals and also the linear or branched positional isomers thereof: alkyl radicals containing from 1 to 6 carbon atoms and more particularly alkyl radicals containing from 1 to 4 carbon atoms of the above list are preferred;
the term “alkoxy radical” denotes linear and, where appropriate, branched methoxy, ethoxy, propoxy, isopropoxy, linear, secondary or tertiary butoxy, pentoxy or hexoxy radicals and also the linear or branched positional isomers thereof: alkoxy radicals containing from 1 to 4 carbon atoms of the above list are preferred;
the term “halogen atom” denotes chlorine, bromine, iodine or fluorine atoms, and preferably the chlorine, bromine or fluorine atom;
the term “cycloalkyl radical” denotes a saturated carbocyclic radical containing 3 to 10 carbon atoms and thus denotes in particular cyclopropyl, cyclobutyl, cyclopentyl and cyclohexyl radicals, and most particularly cyclopropyl, cyclopentyl and cyclohexyl radicals;
the term “heterocycloalkyl radical” thus denotes a monocyclic or bicyclic carbocyclic radical containing from 3 to 10 ring members, interrupted with one or more heteroatoms, which may be identical or different, chosen from oxygen, nitrogen or sulphur atoms: mention may, for example, be made of morpholinyl, thiomorpholinyl, homomorpholinyl, aziridyl, azetidyl, piperazinyl, piperidyl, homopiperazinyl, pyrrolidinyl, imidazolidinyl, pyrazolidinyl, tetrahydrofuryl, tetrahydrothienyl, tetrahydropyranyl, tetrahydropyran, oxodihydropyridazinyl or else oxetanyl radicals, all these radicals being optionally substituted; mention may in particular be made of tetrahydropyranyl, morpholinyl, thiomorpholinyl, homomorpholinyl, piperazinyl, piperidyl, homopiperazinyl or else pyrrolidinyl radicals;
the term “—O-heterocycloalkyl radical” denotes a heterocycloalkyl radical as defined above, bearing an —O— (oxy) function: mention may, for example, be made of morpholinyloxy, thiomorpholinyloxy, homomorpholinyloxy, aziridyloxy, azetidyloxy, piperazinyloxy, piperidyloxy, homopiperazinyloxy, pyrrolidinyloxy, imidazolidinyloxy, pyrazolidinyloxy, tetrahydrofuryloxy, tetrahydrothienyloxy, tetrahydropyranyloxy, hexahydropyrannoxy, oxodihydropyridazinyloxy or else oxetanyloxy radicals, all these radicals being optionally substituted; mention may in particular be made of tetrahydro-2H-pyran-2-yloxy, morpholinyloxy, thiomorpholinyloxy, homomorpholinyloxy, piperazinyloxy, piperidyloxy, homopiperazinyloxy or else pyrrolidinyloxy radicals;
the terms “aryl” and “heteroaryl” denote monocyclic or bicyclic, unsaturated or partially unsaturated, respectively carbocyclic and heterocyclic radicals containing at most 12 ring members, which may optionally contain a —C(O) ring member, the heterocyclic radicals containing one or more heteroatoms, which may be identical or different, chosen from O, N or S with N, where appropriate, optionally substituted;
the term “aryl radical” thus denotes monocyclic or bicyclic radicals containing 6 to 12 ring members, such as, for example, phenyl, naphthyl, biphenyl, indenyl, fluorenyl and anthracenyl radicals, more particularly phenyl and naphthyl radicals, and even more particularly the phenyl radical. It may be noted that a carbocyclic radical containing a —C(O) ring member is, for example, the tetralone radical;
the term “heteroaryl radical” thus denotes monocyclic or bicyclic radicals containing 5 to 12 ring members: monocyclic heteroaryl radicals, for instance the radicals: thienyl such as 2-thienyl and 3-thienyl, furyl such as 2-furyl or 3-furyl, pyrannyl, pyrrolyl, pyrrolinyl, pyrazolinyl, imidazolyl, pyrazolyl, pyridyl such as 2-pyridyl, 3-pyridyl and 4-pyridyl, pyrazinyl, pyrimidinyl, pyridazinyl, oxazolyl, thiazolyl, isothiazolyl, diazolyl, thiadiazolyl, thiatriazolyl, oxadiazolyl, isoxazolyl such as 3- or 4-isoxazolyl, furazanyl or tetrazolyl, which may be free or salified, all these radicals being optionally substituted, among which more particularly the radicals: thienyl such as 2-thienyl and 3-thienyl, furyl such as 2-furyl, pyrrolyl, pyrrolinyl, pyrazolinyl, imidazolyl, pyrazolyl, oxazolyl, isoxazolyl, pyridyl, pyridazinyl, these radicals being optionally substituted; bicyclic heteroaryl radicals, for instance the radicals: benzothienyl such as 3-benzothienyl, benzothiazolyl, quinolyl, isoquinolyl, dihydroquinolyl, quinolone, tetralone, adamentyl, benzofuryl, isobenzofuryl, dihydrobenzofuran, ethylenedioxyphenyl, thianthrenyl, benzopyrrolyl, benzimidazolyl, benzoxazolyl, thionaphthyl, indolyl, azaindolyl, indazolyl, purinyl, thienopyrazolyl, tetrahydroindazolyl, tetrahydrocyclopentapyrazolyl, dihydrofuropyrazolyl, tetrahydropyrrolopyrazolyl, oxotetrahydropyrrolo-pyrazolyl, tetrahydropyranopyrazolyl, tetrahydropyridinopyrazolyl or oxodihydropyridinopyrazolyl, all these radicals being optionally substituted.

As examples of heteroaryl or bicyclic radicals, mention may more particularly be made of pyrimidinyl, pyridyl, pyrrolyl, azaindolyl, indazolyl, pyrazolyl, benzothiazolyl or benzimidazolyl radicals, optionally substituted with one or more substituents, which may be identical or different, as indicated above.

The carboxyl radical(s) of the products of formula (I) may be salified or esterified with the various groups known to those skilled in the art, among which mention may, for example, be made of:
among the salification compounds, mineral bases such as, for example, an equivalent of sodium, of potassium, of lithium, of calcium, of magnesium or of ammonium or organic bases such as, for example, methylamine, propylamine, trimethylamine, diethylamine, triethylamine, N,N-dimethyl-ethanolamine, tris(hydroxymethyl)aminomethane, ethanolamine, pyridine, picoline, dicyclohexylamine, morpholine, benzylamine, procaine, lysine, arginine, histidine or N-methylglucamine,
among the esterification compounds, alkyl radicals for forming alkoxycarbonyl groups, such as, for example, methoxycarbonyl, ethoxycarbonyl, tert-butoxycarbonyl or benzyloxycarbonyl, these alkyl radicals possibly being substituted with radicals chosen, for example, from halogen atoms, and hydroxyl, alkoxy, acyl, acyloxy, alkylthio, amino or aryl radicals, such as for instance in chloromethyl, hydroxypropyl, methoxymethyl, propionyloxymethyl, methylthiomethyl, dimethylaminoethyl, benzyl or phenethyl groups.

The addition salts with mineral or organic acids of the products of formula (I) may, for example, be the salts formed with hydrochloric acid, hydrobromic acid, hydroiodic acid, nitric acid, sulphuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxylic acid, aspartic acid, ascorbic acid, alkylmonosulphonic acids such as, for example, methanesulphonic acid, ethanesulphonic acid or propanesulphonic acid, alkyldisulphonic acids such as, for example, methanedisulphonic acid or alpha,beta-ethanedisulphonic acid, arylmonosulphonic acids such as benzenesulphonic acid and aryldisulphonic acids.

It may be recalled that stereoisomerism can be defined in its broad sense as the isomerism of compounds having the same structural formulae, but the various groups of which are arranged differently in space, such as in particular in monosubstituted cyclohexanes, the substituent of which can be in the axial or equatorial position, and the various possible rotational conformations of ethane derivatives. However, another type of stereoisomerism exists, due to the different spatial arrangements of substituents attached either on double bonds or on rings, which is commonly known as geometrical isomerism or cis-trans isomerism. The term “stereoisomers” is used in the present application in its broadest sense and therefore relates to all the compounds indicated above.

When NR1R2 or NR3R4 forms a ring as defined above, such an aminated ring may be chosen, in particular, from pyrrolidinyl, pyrazolidinyl, pyrazolinyl, piperidyl, azepinyl, morpholinyl, homomorpholinyl, piperazinyl or homopiperazinyl radicals, these radicals being themselves optionally substituted as indicated above or hereinafter: for example, with one or more radicals, which may be identical or different, chosen from halogen atoms and alkyl, hydroxyl, alkoxy, phenyl and CH₂-phenyl radicals, the alkyl or phenyl radicals being themselves optionally substituted with one or more radicals, which may be identical or different, chosen from halogen atoms and alkyl, hydroxyl, alkoxy, NH₂, NHalk and N(alk)₂ radicals.

The NR1R2 or NR3R4 ring may more particularly be chosen from pyrrolidinyl radicals or morpholino radicals, optionally substituted with one or two alkyl radicals or piperazinyl radicals, optionally substituted on the second nitrogen atom with an alkyl, phenyl, or CH₂-phenyl radical, themselves optionally substituted with one or more radicals, which may be identical or different, chosen from halogen atoms and alkyl, hydroxyl and alkoxy radicals.

A subject of the present invention is the products of formula (I) as defined above or hereinafter, in which:

Ra represents a hydrogen atom; an iodine atom; or a phenyl radical optionally substituted with a halogen atom;
Rb represents a hydrogen atom, a CO-Rc radical or a —CO—NRcRd radical;
where Rc represents a cyclopropyl radical or an alkyl radical optionally substituted with an alkoxy or NR1R2 radical;
Rd represents a hydrogen atom;
NR1R2 being such that: either R1 and R2, which may be identical or different, represent a hydrogen atom or an alkyl radical; or R1 and R2 form, with the nitrogen atom to which they are attached, a morpholinyl or piperazinyl radical optionally substituted on the second nitrogen atom with an alkyl radical;
the alkyl or alkoxy radicals above containing from 1 to 4 carbon atoms;
said products of formula (I) being in all the possible racemic, enantiomeric and diastereoisomeric isomer forms, and also the addition salts with inorganic and organic acids or with inorganic and organic bases of said products of formula (I).

A subject of the present invention is most particularly the products of formula (I) as defined above, corresponding to the following formulae:
N-[6-([1,2,4]triazolo[4,3-a]pyridin-3-ylsulphanyl)-1,3-benzothiazol-2-yl]cyclopropanecarboxamide
1-[2-(morpholin-4-yl)ethyl]-3-[6-([1,2,4]triazolo[4,3-a]pyridin-3-ylsulphanyl)-1,3-benzothiazol-2-yl]urea
1-[2-(4-methylpiperazin-1-yl)ethyl]-3-[6-([1,2,4]triazolo[4,3-a]pyridin-3-ylsulphanyl)-1,3-benzothiazol-2-yl]urea
1-(2-methoxyethyl)-3-[6-([1,2,4]triazolo[4,3-a]pyridin-3-ylsulphanyl)-1,3-benzothiazol-2-yl]urea
6-[(6-iodo[1,2,4]triazolo[4,3-a]pyridin-3-yl)sulphanyl]-1,3-benzothiazol-2-amine
6-{[6-(4-fluorophenyl)[1,2,4]triazolo[4,3-a]pyridin-3-yl]sulphanyl}-1,3-benzothiazol-2-amine
N-{6-[6-(4-fluorophenyl)[1,2,4]triazolo[4,3-a]pyridin-3-ylsulphanyl]-1,3-benzothiazol-2-yl}cyclopropanecarboxamide
6-{[6-(1-methyl-1H-pyrazol-4-yl)[1,2,4]triazolo[4,3-a]pyridin-3-yl]sulphanyl}-1,3-benzothiazol-2-amine
N-(6-{[6-(1-methyl-1H-pyrazol-4-yl)[1,2,4]triazolo[4,3-a]pyridin-3-yl]sulphanyl}-1,3-benzothiazol-2-yl)cyclopropanecarboxamide
N-(6-{[6-(1H-pyrazol-4-yl)[1,2,4]triazolo[4,3-a]pyridin-3-yl]sulphanyl}-1,3-benzothiazol-2-yl)cyclopropanecarboxamide
N-(6-{[6-((3-fluoro-4-methyl)phenyl)[1,2,4]triazolo[4,3-a]pyridin-3-yl]sulphanyl}-1,3-benzothiazol-2-yl)cyclopropanecarboxamide
N-(6-{[6-(3-fluorophenyl)[1,2,4]triazolo[4,3-a]pyridin-3-yl]sulphanyl}-1,3-benzothiazol-2-yl)cyclopropanecarboxamide
N-(6-{[6-(1-[2-(tetrahydro-2H-pyran-2-yloxy)ethyl]-1H-pyrazol-4-yl)[1,2,4]triazolo[4,3-a]pyridin-3-yl]sulphanyl}-1,3-benzothiazol-2-yl)cyclopropanecarboxamide
N-(6-{[6-(1-(2-hydroxyethyl)-1H-pyrazol-4-yl)[1,2,4]triazolo[4,3-a]pyridin-3-yl]sulphanyl}-1,3-benzothiazol-2-yl)cyclopropanecarboxamide
N-(6-{[6-(1-piperidin-4-yl-1H-pyrazol-4-yl)[1,2,4]triazolo[4,3-a]pyridin-3-yl]sulphanyl}-1,3-benzothiazol-2-yl)cyclopropanecarboxamide
   and also the addition salts with inorganic and organic acids or with inorganic and organic bases of said products of formula (I).

A subject of the present invention is also any process for preparing the products of formula (I) as defined above.

The products according to the invention can be prepared using conventional organic chemistry methods.

### Preparation of Compounds of Formula (I)

Schemes 1, 2 and 3 below illustrate the methods used to prepare the products of formula (I). In this respect, they cannot constitute a limitation of the scope of the invention, with regard to the methods for preparing the compounds claimed.

The products of formula (I) as defined above according to the present invention may thus in particular be prepared according to the process described in schemes 1, 2 and 3 below.

A subject of the present invention is thus also the process for preparing products of formula (I) according to scheme 1 as defined hereinafter.

A subject of the present invention is thus also the process for preparing products of formula (I) according to scheme 2 as defined hereinafter.

A subject of the present invention is thus also the process for preparing products of formula (I) according to scheme 3 as defined hereinafter.

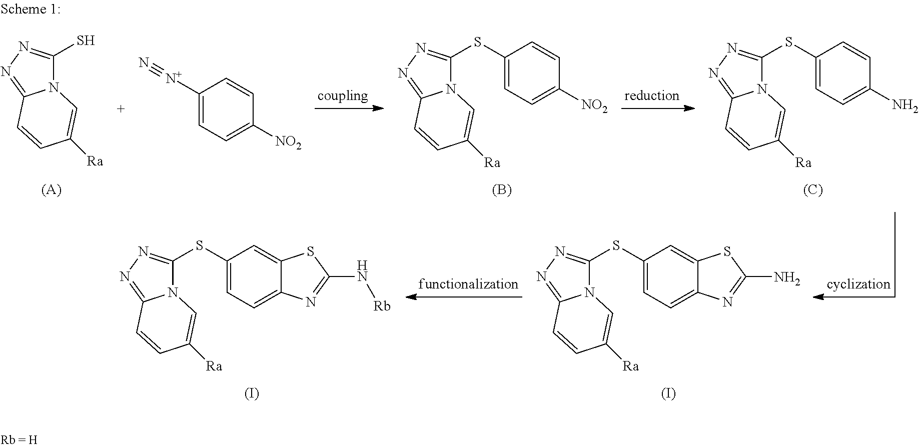

In scheme 1 above, the substituents Ra and Rb have the meanings indicated above.

The compounds (I) for which Ra and Rb have the same meanings can be obtained from the compounds (I) for which Rb=H.

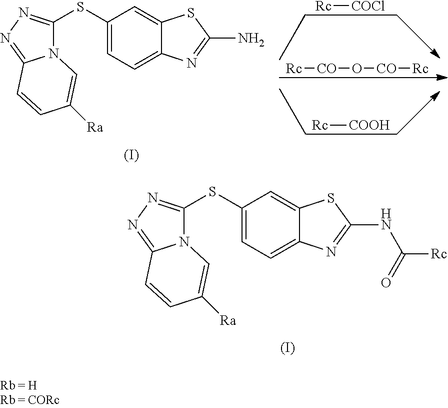

More particularly, the compounds (I) for which Rb=CORc (with Rc as defined above) can be obtained, for example:

by reacting an acid chloride of formula Rc-COCl in the presence, for example, of a solvent such as pyridine at a temperature in the region of 20° C.,
by reacting an acid anhydride of formula Rc-CO—O—CO—Rc, in the presence, for example, of a solvent such as pyridine at a temperature in the region of 20° C.,
by reacting with a carboxylic acid of formula Rc-COOH under the conditions, for example, described by D. DesMarteau et al. (Chem. Lett., 2000, 9, 1052) in the presence of 1-hydroxybenzotriazole and of 1-(3-dimethylaminopropyl)-3-ethylcarbodiimide and in the presence of a base such as triethylamine, at a temperature between 20° C. and the reflux temperature of the solvent.

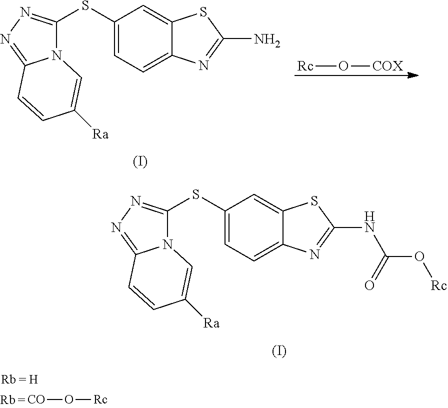

More particularly, the compounds (I) for which Rb=CO—O-Rc (with Rc as defined above) can be obtained, for example, by reaction with a chlorocarbonate Rc-O—COX (X=Cl) on the compounds (I) for which Rb=H, in a solvent such as tetrahydrofuran, in the presence of a base such as sodium hydrogen carbonate, or in pyridine, at a temperature in the region of 20° C.

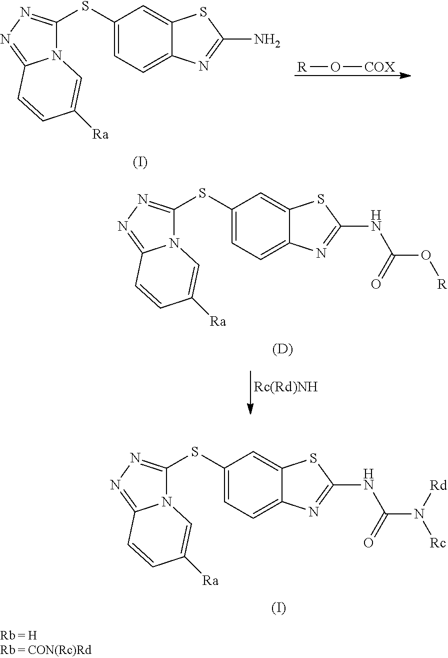

More particularly, the compounds (I) for which Rb=CON(Rc)Rd (with Rc and Rd as defined above) can be obtained, for example, by reacting carbamates (D) where R=phenyl, with amines Rc(Rd)NH (with Rc and Rd as defined above), in the presence of an aprotic solvent such as tetrahydrofuran, at a temperature in the region of 20° C.

The carbamates (D) can be obtained, for example, by reaction with a chlorocarbonate R—O—COX (X=Cl) on the compounds (I) for which Rb=H, in a solvent such as tetrahydrofuran, in the presence of a base such as sodium hydrogen carbonate, or in pyridine, at a temperature in the region of 20° C.

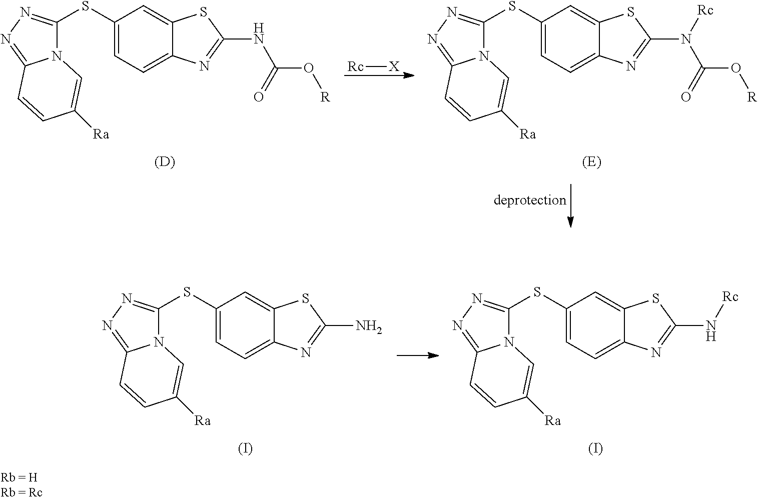

More particularly, the compounds (I) for which Rb=Rc (with Rc as defined above) can be obtained, for example:
by deprotection of the carbamates (E) with R=t-butyl according to a customary method for those skilled in the art, for example with trifluoroacetic acid, in a solvent such as dichloromethane at a temperature in the region of 20° C.;
from the compounds (I) for which Rb=H, by application of the methods described in patent EP 0408437 or by R. A Glennon et al. (Journal of Medicinal Chemistry, 1981, 24, 766-769).

The carbamates (E) can be obtained, for example, by reacting the carbamates (D) where R=t-butyl, with halides Rc-X (with Rc as defined above), in the presence of a solvent such as N,N-dimethylformamide, in the presence of a base such as sodium hydride, at a temperature of between 20° C. and 90° C.

The compounds (I) for which Rb=H can be obtained by cyclization of the compounds (C) according to a customary method for those skilled in the art, for example by application of the methods described by H. Masaichi et al. (Journal of Medicinal Chemistry, 2007, 50(18), 4453-4470), by reacting potassium thiocyanate and bromine in the presence of an acid such as acetic acid, at a temperature of between 20° C. and the reflux temperature of the solvent.

The compounds (C) can be obtained by reduction of the compounds (B) according to a customary method for those skilled in the art, for example using tin chloride in a solvent such as ethanol, or alternatively using hydrogen in the presence of a catalyst, such as palladium-on-charcoal or Raney nickel.

The compounds (B) can be obtained by coupling the compounds A), with Ra as defined above, with 4-nitrobenzenediazonium tetrafluoroborate (commercial product), under the conditions described, for example, by M. A. Biamonte et al. (Journal of Organic Chemistry, 2005, 70, 717-720), possibly in the presence of a base such as sodium hydrogen carbonate, for example in a solvent such as dimethyl sulphoxide, acetone or acetonitrile, at a temperature of between 20° C. and the reflux temperature of the solvent.

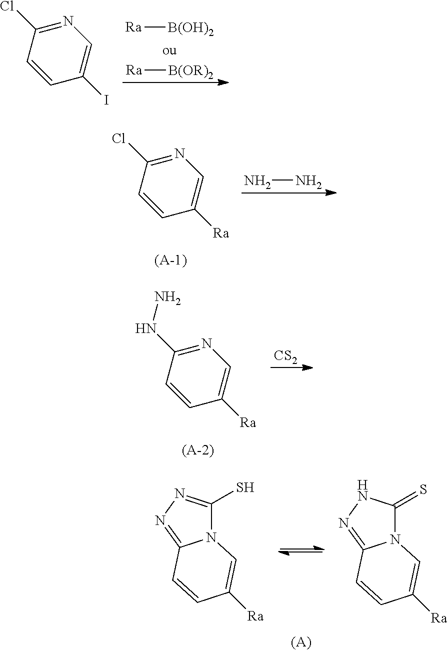

The compounds (A) are either commercially available, or prepared by application of the methods described in patent EP 0254623 or in U.S. Pat. No. 4,244,953, using the hydrazino derivatives of formula (A2), by reaction with carbon disulphide in a solvent such as pyridine or chloroform at a temperature of between 20° C. and the reflux of the solvent.

The compounds (A2) are either commercially available, or obtained by application of the methods described in patent EP 0254623, in U.S. Pat. No. 4,244,953 or according to R. Church et al. (Journal of Organic Chemistry 1995, 60, 3750-3758) using the 2-chloropyridine derivatives (A1), by reaction of hydrazine or hydrazine hydrate.

The compounds (A1) are either commercially available, or can be obtained using 2-chloro-5-iodopyridine (commercial compound), for example:

using boronic acids of formula Ra—B(OH)₂ in the presence of potassium phosphate and of tetrakis(triphenylphosphine)palladium, in a solvent such as dimethyl sulphoxide, at a temperature in the region of 80° C., or using the boronic esters Ra—B(OR)₂ in the presence of dichlorobis(triphenylphosphine)-palladium in a solvent such as, for example, 1,2-dimethoxyethane, in the presence of a base such as 1N sodium hydroxide, at a temperature in the region of 80° C.

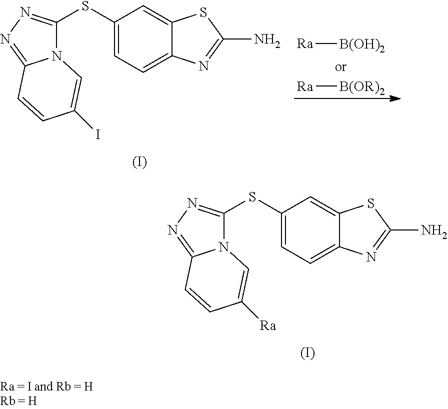

The compounds (I) for which Rb=H can also be obtained from the compound (I) for which Rb=H and Ra=I by reaction of the boronic acids of formula Ra—B(OH)₂ or by reaction of the boronic esters Ra—B(OR)₂ as described for the preparation of the compounds (A1).

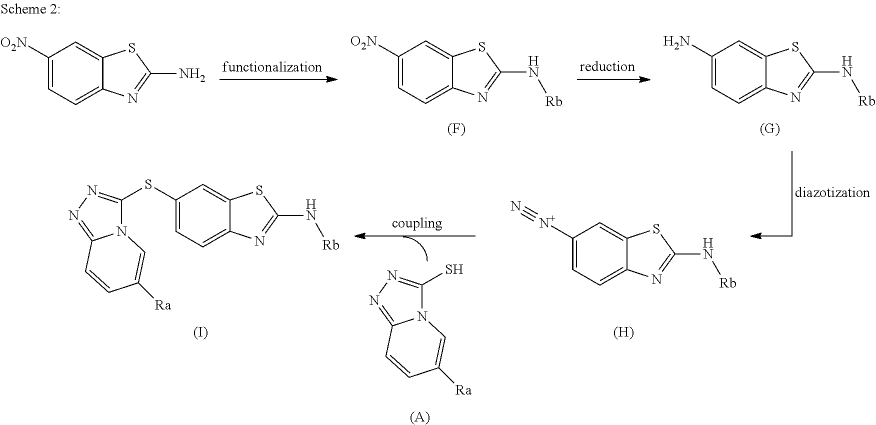

In scheme 2 above, the substituents Ra and Rb have the meanings indicated above.

The compounds (I) for which Ra and Rb have the same meanings indicated above can be obtained by coupling reaction of the compounds (A) with Ra as defined above, with the compounds (H) with Rb as defined above, as described for the preparation of the compounds (B) above.

The compounds (H) for which Rb has the same meanings indicated above can be obtained by diazotization of the compounds (G) according to a customary method for those skilled in the art, for example, by reaction of nitrous acid (HNO₂) or of sodium nitrite (NaNO₂) in the presence of an acid such as aqueous tetrafluoroboric acid, at a temperature in the region of 20° C.

The compounds (G) for which Rb has the same meanings indicated above can be obtained by reduction of the compounds (F) according to a customary method for those skilled in the art, for example, using hydrogen in the presence of a catalyst such as palladium-on-charcoal or Raney nickel, in a solvent such as tetrahydrofuran, for example, at a temperature of between 20° C. and the reflux of the solvent.

The compounds (F) for which Rb has the same meanings indicated above can be obtained from 2-amino-6-nitrobenzothiazole (commercial product) as described above for the preparation of the compounds (I) from the compounds (I) for which Rb=H.

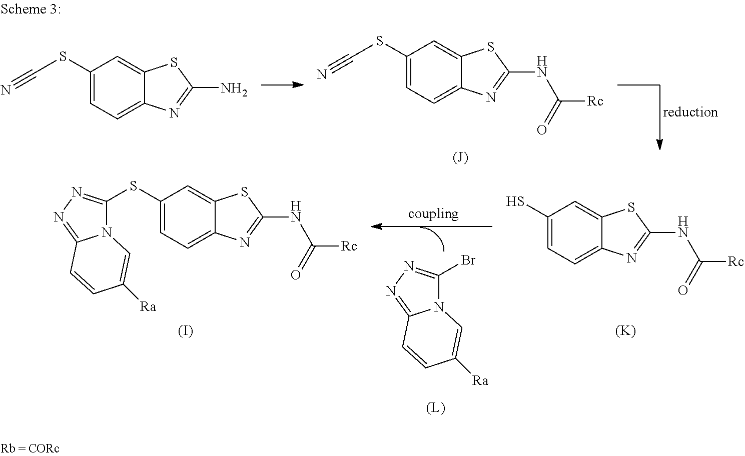

In scheme 3 above, the substituents Ra and Rc have the meanings indicated above.

The compounds (I) for which Ra has the same meanings as above and for which Rb=CORc can be obtained by coupling reaction of the compounds (L), with Ra as defined above, with the compounds (K), with Rc as defined above, under the conditions described, for example, by R. Varala et al. (Chemistry Letters, 2004, 33(12), 1614-1615), or by M. Winn et al. (Journal of Medicinal Chemistry, 2001, 44, 4393-4403), in the presence of a base such as, for example, potassium carbonate, in a solvent such as dimethyl sulphoxide, at a temperature of between 20° C. and the reflux temperature of the solvent. Such reactions can also be carried out under microwaves.

The compounds (K) for which Rc has the same meanings indicated above can be obtained, for example, by reduction of the compounds (J) with DL-dithiotreitol, in the presence of sodium hydrogen carbonate or of potassium dihydrogen phosphate, in a solvent such as ethanol and at a temperature of between 20° C. and the reflux of the solvent.

The compounds (J) for which Rc has the same meanings indicated above can be obtained from 2-amino-1,3-benzothiazol-6-yl thiocyanate (commercial product) as described above for the preparation of the compounds (I) with Rb=CORc, from the compounds (I) with Rb=H.

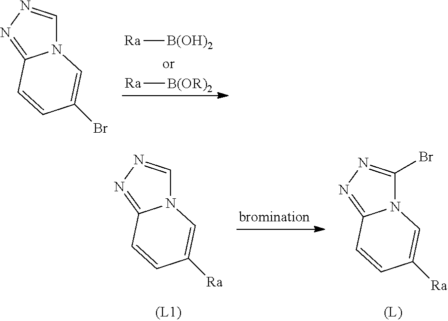

The compounds (L) are either commercially available (Ra=H), or prepared by bromination of the compounds (L1), according to a customary method for those skilled in the art, for example according to the conditions described by E. S. Hand et al. (Journal of Organic Chemistry, 1980, 45, 3738-3745) or using bromine in a solvent such as ethanol at a temperature of between 20° C. and the reflux of the solvent.

The compounds (L1) are either commercially available (Ra=H), or can be obtained using 6-bromo[1,2-4]triazolo[4,3-a]pyridine (commercial product), by coupling reaction, by application of the methods described by C. Enguehard et al. (Helvetica Chimica Acta (2001), 84, 3610-3614), for example:
using the boronic acids of formula Ra—B(OH)₂ in the presence of sodium hydrogen carbonate and of tetrakis(triphenylphosphine)palladium in a solvent such as dimethyl sulphoxide or dioxane, at a temperature in the region of 80° C.,
using the boronic esters Ra—B(OR)₂ in the presence of dichlorobis(triphenylphosphine)palladium in a solvent such as, for example, 1,2-dimethoxyethane, in the presence of a base such as 1N sodium hydroxide, at a temperature in the region of 80° C.

Among the starting products of formula (e) (A), (A1), (A2), (F), (G), (L) and (L1), some are known and can be obtained either commercially, or according to the usual methods known to those skilled in the art, for example starting from commercial products.

It is understood, for those skilled in the art, that, in order to carry out the processes according to the invention described above, it may be necessary to introduce protective groups for amino, carboxyl and alcohol functions in order to avoid side reactions.

The following non-exhaustive list of examples of protection of reactive functions may be mentioned:
hydroxyl groups may be protected, for example, with alkyl radicals such as tert-butyl, trimethysilyl, tert-butyldimethylsilyl, methoxymethyl, tetrahydropyranyl, benzyl or acetyl,
amino groups may be protected, for example, with acetyl, trityl, benzyl, tert-butoxycarbonyl (BOC), benzyloxycarbonyl or phthalimido radicals or other radicals known in peptide chemistry.

Acid functions may be protected, for example, in the form of esters formed with readily cleavable esters such as benzyl or tert-butyl esters or esters known in peptide chemistry.

A list of various protective groups that may be used will be found in the textbooks known to those skilled in the art and, for example, in patent BF 2 499 995.

It may be noted that it is possible, if desired and if necessary, to subject intermediate products or products of formula (I) thus obtained by the processes indicated above, in order to obtain other intermediates or other products of formula (I), to one or more conversion reactions known to those skilled in the art, for instance:

a) a reaction for esterification of an acid function,
b) a reaction for saponification of an ester function to give an acid function,
c) a reaction for reducing a free or esterified carboxyl function to given an alcohol function,
d) a reaction for conversion of an alkoxy function to give a hydroxyl function, or alternatively of a hydroxyl function to give an alkoxy function,
e) a reaction for removal of the protective groups that may be borne by the protected reactive functions,
f) a reaction for salification with an inorganic or organic acid or with a base so as to obtain the corresponding salt,
g) a reaction for resolution of the racemic forms to give resolved products,
said products of formula (I) thus obtained being in all the possible racemic, enantiomeric and diastereoisomeric isomer forms.

The reactions a) to g) can be carried out under the usual conditions known to those skilled in the art, for instance those indicated hereinafter.

a) The products described above may, if desired, undergo, on the possible carboxyl functions, esterification reactions that may be performed according to the usual methods known to those skilled in the art.
b) The possible conversions of ester functions to give acid functions of the products described above may, if desired, be performed under the usual conditions known to those skilled in the art, in particular by acid or alkaline hydrolysis, for example with sodium hydroxide or potassium hydroxide in an alcoholic medium, for instance in methanol, or alternatively with hydrochloric acid or sulphuric acid.

The saponification reaction may be carried out according to the usual methods known to those skilled in the art, for instance in a solvent such as methanol or ethanol, dioxane or dimethoxyethane, in the presence of sodium hydroxide or potassium hydroxide.

c) The possible free or esterified carboxyl functions of the products described above may be reduced, if desired, to give alcohol functions via the methods known to those skilled in the art; the possible esterified carboxyl functions may be reduced, if desired, to give alcohol functions by the methods known to those skilled in the art, and in particular with lithium aluminium hydride in a solvent such as, for example, tetrahydrofuran, or else dioxane or ethyl ether.

The possible free carboxyl functions of the products described above may be reduced, if desired, to give alcohol functions, in particular with boron hydride.

d) The possible alkoxy functions, such as in particular methoxy, of the products described above may be converted, if desired, into hydroxyl functions under the usual conditions known to those skilled in the art, for example with boron tribromide in a solvent such as, for example, methylene chloride, with pyridine hydrochloride or hydrobromide, or alternatively with hydrobromic acid or hydrochloric acid in water or trifluoroacetic acid at reflux.
e) The removal of protective groups, for instance those indicated above, may be carried out under the usual conditions known to those skilled in the art, in particular via an acid hydrolysis performed with an acid such as hydrochloric acid, benzenesulphonic or para-toluenesulphonic acid, formic acid or trifluoroacetic acid, or alternatively via catalytic hydrogenation.

The phthalimido group may be removed with hydrazine.

f) The products described above may, if desired, undergo salification reactions, for example with an inorganic or organic acid or with an inorganic or organic base according to the usual methods known to those skilled in the art: such a salification reaction may be carried out, for example, in the presence of hydrochloric acid, or alternatively of tartaric acid, citric acid or methanesulphonic acid, in an alcohol such as, for example, ethanol or methanol.
g) The possible optically active forms of the products described above may be prepared by resolution of the racemic mixtures according to the usual methods known to those skilled in the art.

The products of formula (I) as defined above and also the addition salts thereof with acids exhibit advantageous pharmacological properties, in particular owing to their kinase-inhibiting properties as indicated above.

The products of the present invention can in particular be used for treating tumours.

The products of the invention may thus also increase the therapeutic effects of commonly used antitumour agents.

These properties justify their therapeutic use, and a subject of the invention is in particular, as medicaments, the products of formula (I) as defined above, said products of formula (I) being in all the possible racemic, enantiomeric and diastereoisomeric isomer forms, and also the addition salts with pharmaceutically acceptable inorganic and organic acids or with pharmaceutically acceptable inorganic and organic bases of said products of formula (I).

A subject of the invention is most particularly, as medicaments, the products corresponding to the following formulae:
N-[6-([1,2,4]triazolo[4,3-a]pyridin-3-ylsulphanyl)-1,3-benzothiazol-2-yl]cyclopropanecarboxamide
1-[2-(morpholin-4-yl)ethyl]-3-[6-([1,2,4]triazolo[4,3-a]pyridin-3-ylsulphanyl)-1,3-benzothiazol-2-yl]urea
1-[2-(4-methylpiperazin-1-yl)ethyl]-3-[6-([1,2,4]triazolo[4,3-a]pyridin-3-ylsulphanyl)-1,3-benzothiazol-2-yl]urea
1-(2-methoxyethyl)-3-[6-([1,2,4]triazolo[4,3-a]pyridin-3-ylsulphanyl)-1,3-benzothiazol-2-yl]urea
6-[(6-iodo[1,2,4]triazolo[4,3-a]pyridin-3-yl)sulphanyl]-1,3-benzothiazol-2-amine
6-{[6-(4-fluorophenyl)[1,2,4]triazolo[4,3-a]pyridin-3-yl]sulphanyl}-1,3-benzothiazol-2-amine
N-{6-[6-(4-fluorophenyl)[1,2,4]triazolo[4,3-a]pyridin-3-ylsulphanyl]-1,3-benzothiazol-2-yl}cyclopropanecarboxamide
6-{[6-(1-methyl-1H-pyrazol-4-yl)[1,2,4]triazolo[4,3-a]pyridin-3-yl]sulphanyl}-1,3-benzothiazol-2-amine
N-(6-{[6-(1-methyl-1H-pyrazol-4-yl)[1,2,4]triazolo[4,3-a]pyridin-3-yl]sulphanyl}-1,3-benzothiazol-2-yl)cyclopropanecarboxamide
N-(6-{[6-(1H-pyrazol-4-yl)[1,2,4]triazolo[4,3-a]pyridin-3-yl]sulphanyl}-1,3-benzothiazol-2-yl)cyclopropanecarboxamide
N-(6-{[6-((3-fluoro-4-methyl)phenyl)[1,2,4]triazolo[4,3-a]pyridin-3-yl]sulphanyl}-1,3-benzothiazol-2-yl)cyclopropanecarboxamide
N-(6-{[6-(3-fluorophenyl)[1,2,4]triazolo[4,3-a]pyridin-3-yl]sulphanyl}-1,3-benzothiazol-2-yl)cyclopropanecarboxamide
N-(6-{[6-(1-[2-(tetrahydro-2H-pyran-2-yloxy)ethyl]-1H-pyrazol-4-yl)-[1,2,4]triazolo[4,3-a]pyridin-3-yl]sulphanyl}-1,3-benzothiazol-2-yl)cyclopropanecarboxamide
N-(6-{[6-(1-(2-hydroxyethyl)-1H-pyrazol-4-yl)[1,2,4]triazolo[4,3-a]pyridin-3-yl]sulphanyl}-1,3-benzothiazol-2-yl)cyclopropanecarboxamide
N-(6-{[6-(1-piperidin-4-yl-1H-pyrazol-4-yl)[1,2,4]triazolo[4,3-a]pyridin-3-yl]sulphanyl}-1,3-benzothiazol-2-yl)cyclopropanecarboxamide
   and also the addition salts with pharmaceutically acceptable inorganic and organic acids or with pharmaceutically acceptable inorganic and organic bases of said products of formula (I).

The invention also relates to pharmaceutical compositions containing, as active ingredient, at least one of the products of formula (I) as defined above or a pharmaceutically acceptable salt of this product or a prodrug of this product and, where appropriate, a pharmaceutically acceptable carrier.

The invention thus covers the pharmaceutical compositions containing, as active ingredient, at least one of the medicaments as defined above.

Such pharmaceutical compositions of the present invention may also, where appropriate, contain active ingredients of other antimitotic medicaments, such as, in particular, those based on taxol, cisplatin, DNA intercalating agents, and the like.

These pharmaceutical compositions may be administered orally, parenterally or locally by topical application to the skin and the mucous membranes or by intravenous or intramuscular injection.

These compositions may be solid or liquid and may be in any of the pharmaceutical forms commonly used in human medicine, for instance simple or sugar-coated tablets, pills, lozenges, gel capsules, drops, granules, injectable preparations, ointments, creams or gels; they are prepared according to the usual methods. The active ingredient may, therein, be incorporated into excipients normally used in these pharmaceutical compositions, such as talc, gum arabic, lactose, starch, magnesium stearate, cocoa butter, aqueous or nonaqueous carriers, fatty substances of animal or plant origin, paraffin derivatives, glycols, various wetting agents, dispersants or emulsifiers, and preservatives.

The usual dosage, which is variable depending on the product used, the individual treated and the condition in question, may, for example, be from 0.05 to 5 g per day in adults, or preferably from 0.1 to 2 g per day.

A subject of the present invention is also the use of the products of formula (I) as defined above or of pharmaceutically acceptable salts of these products, for the preparation of a medicament for use in inhibiting the activity of a protein kinase.

A subject of the present invention is also the use of products of formula (I) as defined above, for the preparation of a medicament for use in the treatment or prevention of a disease characterized by dysregulation of the activity of a protein kinase.

Such a medicament may in particular be for use in the treatment or prevention of a disease in a mammal.

A subject of the present invention is also the use as defined above, in which the protein kinase is a protein tyrosine kinase.

A subject of the present invention is also the use as defined above, in which the protein tyrosine kinase is MET or mutant forms thereof.

A subject of the present invention is also the use as defined above, in which the protein kinase is in a cell culture.

A subject of the present invention is also the use as defined above, in which the protein kinase is in a mammal.

A subject of the present invention is in particular the use of a product of formula (I) as defined above, for the preparation of a medicament for use in the prevention or treatment of diseases associated with an uncontrolled proliferation.

A subject of the present invention is in particular the use of a product of formula (I) as defined above, for the preparation of a medicament for use in the treatment or prevention of a disease chosen from the following group: blood vessel proliferation disorders, fibrotic disorders, ‘mesangial’ cell proliferation disorders, metabolic disorders, allergies, asthma, thrombosis, nervous system diseases, retinopathy, psoriasis, rheumatoid arthritis, diabetes, muscle degeneration and cancers.

A subject of the present invention is thus most particularly the use of a product of formula (I) as defined above, for the preparation of a medicament for use in the treatment or prevention of diseases in oncology, and in particular for use in the treatment of cancers.

Among these cancers, the treatment of solid or liquid tumours and the treatment of cancers that are resistant to cytotoxic agents are of interest.

The cited products of the present invention may in particular be used for the treatment of primary tumours and/or metastases, in particular gastric, hepatic, renal, ovarian, colon, prostate and lung (NSCLC and SCLC) cancers, glioblastomas, thyroid, bladder or breast cancers, in melanomas, in lymphoid or myeloid hematopoietic tumours, in sarcomas, in brain, larynx or lymphatic system cancers, bone cancers and pancreatic cancers.

A subject of the present invention is also the use of the products of formula (I) as defined above, for the preparation of medicaments for use in cancer chemotherapy.

Such medicaments for use in cancer chemotherapy may be used alone or in combination.

The products of the present invention may in particular be administered alone or in combination with chemotherapy or radiotherapy or alternatively in combination, for example, with other therapeutic agents.

Such therapeutic agents may be commonly used antitumour agents.

As kinase inhibitors, mention may be made of butyrolactone, flavopiridol and 2-(2-hydroxyethylamino)-6-benzylamino-9-methylpurine, also known as olomoucine.

A subject of the present invention is also, as novel industrial products, the synthesis intermediates of formulae (A), (B), (C), (D), (E), (H), (L), (L1), (J) and (K) as defined above and recalled hereinafter:

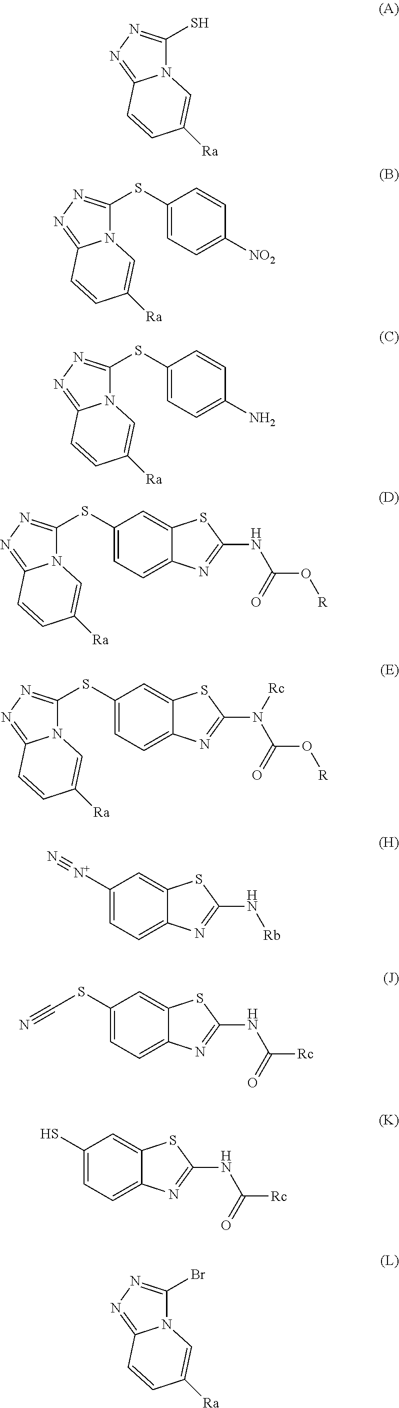

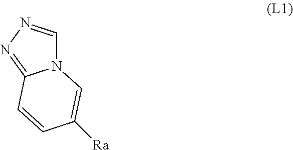

in which Ra, Rb and Rc have the meanings indicated above and R represents a t-butyl or phenyl radical.

The following examples, which are products of formula (I), illustrate the invention without, however, limiting it.

### EXPERIMENTAL SECTION

The nomenclature of the compounds of the present invention was carried out with the ACDLABS software version 10.0.

The ¹H NMR spectra at 400 MHz were acquired on a Bruker Avance DRX-400 spectrometer with the chemical shifts (δ in ppm) in the solvent d₆-dimethyl sulphoxide (DMSO-d₆) referenced at 2.5 ppm at a temperature of 303K.

The infrared (IR) spectra were acquired on a Nicolet Nexus Fourier transform infrared spectrometer; the spectral range is between 4000 and 400 cm⁻¹ with a resolution of 2 cm⁻¹.

The mass spectra (MS) were obtained either by method A or by method B:

### Method A:

Waters HPLC-SQD machine; ionization: positive and/or negative mode electrospray (ES+/−); chromatographic conditions: column: Acquity BEH C₁₈ 1.7 μm-2.1×50 mm; solvents: A: H₂O (0.1% formic acid) B: CH₃CN (0.1% formic acid); column temperature: 50° C.; flow rate: 1 ml/min; gradient (2 min): from 5 to 50% of B in 0.8 min; 1.2 min: 100% of B; 1.85 min: 100% of B; 1.95: 5% of B; retention time=Tr (min).

### Method B:

Waters ZQ machine; ionization: positive and/or negative mode electrospray (ES+/−); chromatographic conditions: column: XBridge C₁₈ 2.5 μm-3×50 mm; solvents: A: H₂O (0.1% formic acid) B: CH₃CN (0.1% formic acid); column temperature: 70° C.; flow rate: 0.9 ml/min; gradient (7 min): from 5 to 100% of B in 5.3 min; 5.5 min: 100% of B; 6.3 min: 5% of B; retention time=Tr (min).

### Example 1

### 6-([1,2,4]triazolo[4,3-a]pyridin-3-ylsulphanyl)-1,3-benzothiazol-2-amine

### Example 1a

### 6-([1,2,4]triazolo[4,3-a]pyridin-3-ylsulphanyl)-1,3-benzothiazol-2-amine

The compound can be prepared in the following way:

1.84 g of potassium thiocyanate are added, in a single step, to a solution of 1.15 g of 4-([1,2,4]triazolo[4,3-a]pyridin-3-ylsulphanyl)aniline in 33 ml of glacial acetic acid. After stirring for approximately 15 minutes, 0.243 ml of bromine diluted in 5 ml of glacial acetic acid are run in, dropwise, while maintaining the temperature at around 20° C. A precipitate gradually forms and the reaction mixture is stirred for approximately 18 hours at a temperature in the region of 20° C. and is then poured into 100 ml of water. The pH is brought to around 8 by adding potassium carbonate. After stirring for 3 hours at a temperature in the region of 20° C., the precipitate is spin-filter-dried and washed with 3 times 20 ml of water, and dried in a desiccator, under reduced pressure, over phosphorus pentoxide. 1.31 g of 6-([1,2,4]triazolo[4,3-a]pyridin-3-ylsulphanyl)-1,3-benzothiazol-2-amine are obtained in the form of a yellow solid.

Melting point: 260-266° C. (Büchi).

MS: method B; [M+H]⁺ m/z=300; [M−H]⁻ m/z=298; Tr=2.38 min.

¹H NMR (400 MHz, DMSO-d₆) δ ppm 7.10 (td, J=6.8, 1.0 Hz, 1H) 7.26 (m, 2H) 7.48 (ddd, J=9.3, 6.8, 1.0 Hz, 1H) 7.61 (broad s, 2H) 7.80 (m, 1H) 7.88 (dt, J=9.3, 1.0 Hz, 1H) 8.48 (dt, J=6.8, 1.0 Hz, 1H).

### Example 1b

### 4-([1,2,4]triazolo[4,3-a]pyridin-3-ylsulphanyl)aniline

The compound can be prepared in the following way:

1.5 g of 3-[(4-nitrophenyl)sulphanyl][1,2,4]triazolo[4,3-a]pyridine are added to a solution of 6.21 g of stannous chloride dihydrate in 8 ml of ethanol. The orange solution obtained is brought to around 60° C. 8.2 ml of a 10N aqueous solution of hydrochloric acid are run in dropwise, at this temperature, and the reaction mixture is stirred for approximately 30 minutes at this same temperature. After a return to a temperature in the region of 20° C., 200 ml of water are added and the pH of the suspension is adjusted to approximately 12 by adding 30% sodium hydroxide. The medium is extracted with 3 times 250 ml of ethyl acetate. The combined organic phases are washed with 3 times 200 ml of water, and 200 ml of a saturated aqueous solution of sodium chloride, dried over magnesium sulphate, filtered, and concentrated under reduced pressure. 1.09 g of 4-([1,2,4]triazolo[4,3-a]pyridin-3-ylsulphanyl)aniline are obtained in the form of a beige solid.

Melting point: 210° C. (Köfler bench)

MS: method A; [M+H]⁺ m/z=243; Tr=0.42 min.

¹H NMR (400 MHz, DMSO-d₆) δ ppm 5.43 (broad s, 2H) 6.50 (d, J=8.5 Hz, 2H) 7.08 (td, J=6.9, 1.0 Hz, 1H) 7.21 (d, J=8.5 Hz, 2H) 7.45 (ddd, J=9.3, 6.9, 1.0 Hz, 1H) 7.84 (dt, J=9.3, 1.0 Hz, 1H) 8.47 (dd, J=6.9, 1.0 Hz, 1H).

### Example 1c

### 3-[(4-nitrophenyl)sulphanyl][1,2,4]triazolo[4,3-a]pyridine

The compound can be prepared in the following way:

1.57 g of 4-nitrobenzenediazonium tetrafluoroborate are added, in small portions, to a solution of 1 g of [1,2,4]triazolo[4,3-a]pyridine-3-thiol in 15 ml of dimethyl sulphoxide. After stirring for 4 days at a temperature in the region of 20° C., the mixture is poured into 100 ml of water. The precipitate is spin-filter-dried, washed with 3 times 20 ml of water, and once with 10 ml of ethanol and ml of diethyl ether, and then air-dried. 1.22 g of 3-[(4-nitrophenyl)sulphanyl][1,2,4]triazolo[4,3-a]pyridine are obtained in the form of a yellow solid.

Melting point: 178-180° C. (Köfler bench)

MS: method B; [M+H]⁺ m/z=273; Tr=3.10 min.

¹H NMR (400 MHz, DMSO-d₆) δ ppm 7.16 (td, J=6.7, 1.1 Hz, 1H) 7.31 (d, J=9.0 Hz, 2H) 7.58 (ddd, J=9.3, 6.8, 1.1 Hz, 1H) 8.01 (dt, J=9.3, 1.1 Hz, 1H) 8.14 (d, J=9.0 Hz, 2H) 8.42 (dt, J=6.8, 1.1 Hz, 1H).

### Example 2

### N-[6-([1,2,4]triazolo[4,3-a]pyridin-3-ylsulphanyl)-1,3-benzo-thiazol-2-yl]cyclopropanecarboxamide

### Example 2a

### N-[6-([1,2,4]triazolo[4,3-a]pyridin-3-ylsulphanyl)-1,3-benzothiazol-2-yl]cyclopropanecarboxamide

The compound can be prepared in the following way:

0.037 ml of cyclopropanecarbonyl chloride is added to a suspension of 0.1 g of 6-([1,2,4]triazolo[4,3-a]pyridin-3-ylsulphanyl)-1,3-benzothiazol-2-amine and of 2 ml of pyridine. After an overnight period at a temperature in the region of 20° C., 0.037 ml of cyclopropanecarbonyl chloride is added. After an overnight period at a temperature in the region of 20° C., 0.037 ml of cyclopropanecarbonyl chloride is again added. After an overnight period at a temperature in the region of 20° C., 10 ml of water are added and the precipitate is spin-filter-dried, washed with 3 times 2 ml of water, 3 times 2 ml of ethanol, twice 2 ml of diethyl ether, and oven-dried at 50° C. under reduced pressure. 0.068 g of N-[6-([1,2,4]triazolo[4,3-a]pyridin-3-ylsulphanylyl)-1,3-benzothiazol-2-yl]cyclopropanecarboxamide is obtained in the form of a solid.

Melting point: 187-190° C. (Köfler bench)

MS: method A; [M+H]⁺ m/z=368; [M−H]⁻ m/z=366; Tr=0.71 min.

¹H NMR (400 MHz, DMSO-d₆) δ ppm 0.94 (m, 4H) 1.96 (m, 1H) 7.10 (td, J=6.8, 1.0 Hz, 1H) 7.35 (dd, J=8.6, 2.2 Hz, 1H) 7.51 (ddd, J=9.3, 6.8, 1.0 Hz, 1H) 7.66 (d, J=8.6 Hz, 1H) 7.91 (broad d, J=9.3 Hz, 1H) 8.05 (d, J=2.0 Hz, 1H) 8.47 (broad d, J=6.8 Hz, 1H) 12.67 (broad s, 1H).

The compound N-[6-([1,2,4]triazolo[4,3-a]pyridin-3-ylsulphanyl)-1,3-benzothiazol-2-yl]cyclopropanecarboxamide can also be prepared in the following way:

100 mg of 2-[(cyclopropylcarbonyl)amino]-1,3-benzothiazole-6-diazonium tetrafluoroborate are added to a suspension of 36.44 mg of [1,2,4]triazolo[4,3-a]pyridine-3-thiol, 20.25 mg of sodium hydrogen carbonate and 2 ml of acetonitrile. After stirring for 6 days at a temperature in the region of 20° C., the mixture is poured into 20 ml of water. The precipitate is spin-filter-dried, washed with twice 10 ml of diethyl ether, and then air-dried. 40 mg of N-[6-([1,2,4]triazolo[4,3-a]pyridin-3-ylsulphanylyl)-1,3-benzothiazol-2-yl]cyclopropanecarboxamide are thus obtained.

### Example 2b

### 2-[(cyclopropylcarbonyl)amino]-1,3-benzothiazole-6-diazonium tetrafluoroborate

The compound can be obtained in the following way:

133.1 mg of sodium nitrite and 1.5 ml of water are added to a solution of 0.5 g of N-(6-amino-1,3-benzothiazol-2-yl)cyclopropanecarboxamide and 2 ml of aqueous tetrafluoroboric acid (solution at 48%). The reaction medium is kept stirring at ambient temperature for 16 hours. The precipitate formed is filtered off, washed with diethyl ether and then air-dried. 566 mg of 2-[(cyclopropyl-carbonyl)amino]-1,3-benzothiazole-6-diazonium tetrafluoroborate are thus obtained in the form of a white solid.

Melting point: 200° C. (Köfler bench)

MS: method A; [M]⁺: m/z=245; [BF₄]⁻: m/z=87; Tr=0.28 min.

IR: 2253 cm⁻¹ (aryl-diazonium cation); 1150-1000 cm⁻¹, 533 and 523 cm⁻¹ (tetrafluoroborate).

### Example 2c

### N-(6-amino-1,3-benzothiazol-2-yl)cyclopropanecarboxamide

The compound can be prepared in the following way:

1.5 g of N-(6-nitro-1,3-benzothiazol-2-yl)cyclopropanecarboxamide, 150 mg of palladium-on-charcoal (10%) and 150 ml of tetrahydrofuran are charged to an autoclave. The medium is then stirred under a hydrogen pressure of 15 bar and heated to 50° C. After a return to normal pressure and to ambient temperature, the medium is filtered through celite and the filtrate is concentrated by evaporation under reduced pressure. 1.3 g of N-(6-amino-1,3-benzothiazol-2-yl)cyclopropanecarboxamide are thus obtained in the form of a white solid.

Melting point>260° C. (Köfler bench)

MS: method A; [M+H]⁺: m/z 234; Tr=0.34 min.

### Example 2d

### N-(6-nitro-1,3-benzothiazol-2-yl)cyclopropanecarboxamide

The compound can be prepared in the following way:

2.3 ml of cyclopropanecarbonyl chloride are added dropwise to a suspension of 5 g of 2-amino-6-nitrobenzothiazole (commercial product) and 50 ml of anhydrous pyridine. The reaction mixture is then kept stirring at ambient temperature for 24 hours. The precipitate formed is filtered off, rinsed with 100 ml of water, twice 10 ml of ethanol and twice 20 ml of diethyl ether, and then spin-filter-dried and air-dried. 5.14 g of N-(6-nitro-1,3-benzothiazol-2-yl)cyclopropanecarboxamide are thus obtained in the form of a white powder.

Melting point>260° C. (Köfler bench)

¹H NMR (400 MHz, DMSO-d₆) δ ppm 0.92-1.05 (m, 4H) 1.97-2.08 (m, 1H) 7.86 (d, J=8.9 Hz, 1H) 8.26 (dd, J=8.9, 2.4 Hz, 1H) 9.01 (d, J=2.4 Hz, 1H) 13.02 (broad m, 1H).

### Example 3

### 1-[2-(morpholin-4-yl)ethyl]-3-[6-([1,2,4]triazolo[4,3-a]pyridin-3-ylsulphanyl)-1,3-benzothiazol-2-yl]urea

### Example 3a

### 1-[2-(morpholin-4-yl)ethyl]-3-[6-([1,2,4]triazolo[4,3-a]pyridin-3-ylsulphanyl)-1,3-benzothiazol-2-yl]urea

The compound can be prepared in the following way:

0.1 ml of 2-(morpholin-4-yl)ethanamine is added to a suspension of 0.3 g of phenyl[6-([1,2,4]triazolo[4,3-a]pyridin-3-ylsulphanyl)-1,3-benzothiazol-2-yl]carbamate in 7 ml of tetrahydrofuran. After an overnight period of stirring at a temperature in the region of 20° C., 0.028 ml of 2-(morpholin-4-yl)ethanamine is added and the reaction mixture is stirred overnight at a temperature in the region of 20° C. The mixture is then poured into 100 ml of dichloromethane. The organic phase is washed with 50 ml of a 2N aqueous sodium hydroxide solution. The aqueous phase is supplemented with glacial acetic acid in order to adjust the pH to around 4, and extracted with 3 times 100 ml of dichloromethane, 3 times 100 ml of ethyl acetate and 3 times 100 ml of n-butanol, and the resulting products are dried over magnesium sulphate, filtered, and concentrated under reduced pressure. A solid is obtained, and is taken up with 20 ml of water, spin-filter-dried, washed with twice 2 ml of water, 3 times 5 ml of acetonitrile and 3 times 5 ml of diethyl ether, and air-dried. 0.13 g of 1-[2-(morpholin-4-yl)ethyl]-3-[6-([1,2,4]triazolo[4,3-a]pyridin-3-ylsulphanyl)-1,3-benzothiazol-2-yl]urea is obtained in the form of a white solid.

Melting point: 204-207° C. (Köfler bench)

MS: method A; [M+H]+ m/z=456; [M+H—C₇H₁₂N₂O₂]+ m/z=300; [C₇H₁₃N₂O₂]+ m/z=157 (base peak); [M−H]− m/z=454; Tr=0.45 min.

¹H NMR (400 MHz, DMSO-d₆) δ ppm 2.35-2.44 (m, 6H) 3.25 (m partially masked, 2H) 3.58 (m, 4H) 6.88 (broad m, 1H) 7.11 (broad d, J=6.8 Hz, 1H) 7.29 (dd, J=8.3, 2.0 Hz, 1H) 7.44-7.53 (m, 2H) 7.90 (broad d, J=9.3 Hz, 1 H) 7.95 (broad s, 1H) 8.48 (broad d, J=6.8 Hz, 1H) 11.23 (broad m, 1H).

### Example 3b

### Phenyl[[6-([1,2,4]triazolo[4,3-a]pyridin-3-ylsulphanyl)-1,3-benzothiazol-2-yl]carbamate

The compound can be prepared in the following way:

1.68 ml of phenyl chlorocarbonate and then 2.7 ml of water and 1.12 g of sodium hydrogen carbonate are added to a suspension of 1 g of 6-[1,2,4]triazolo[4,3-a]pyridin-3-ylsulphanyl)-1,3-benzothiazol-2-amine in 27 ml of tetrahydrofuran. The mixture is stirred at a temperature in the region of 20° C. for approximately 48 hours. The precipitate is spin-filter-dried, washed with 10 ml of tetrahydrofuran containing 10% of water and 3 times with 10 ml of ethyl acetate, and air-dried. 0.59 g of phenyl[[6-([1,2,4]triazolo[4,3-a]pyridin-3-ylsulphanyl)-1,3-benzothiazol-2-yl]carbamate is obtained.

MS: method B; [M+H]+ m/z=420; [M−H]− m/z=418; Tr=3.71 min.

¹H NMR (400 MHz, DMSO-d₆) δ ppm 7.11 (td, J=6.8, 1.0 Hz, 1H) 7.25-7.33 (m, 3H) 7.36 (dd, J=8.5, 2.0 Hz, 1H) 7.45 (t, J=7.8 Hz, 2H) 7.51 (ddd, J=9.3, 6.8, 1.0 Hz, 1H) 7.66 (d, J=8.5 Hz, 1H) 7.91 (broad d, J=9.3 Hz, 1H) 8.05 (broad d, J=2.0 Hz, 1H) 8.48 (broad d, J=6.8 Hz, 1H) 12.68 (broad m, 1H).

### Example 4

### 1-[2-(4-methylpiperazin-1-yl)ethyl]-3-[6-([1,2,4]triazolo[4,3-a]pyridin-3-ylsulphanyl)-1,3-benzothiazol-2-yl]urea

The compound can be prepared as in Example 3a, but using 0.2 g of phenyl [[6-([1,2,4]triazolo[4,3-a]pyridin-3-ylsulphanyl)-1,3-benzothiazol-2-yl]carbamate and 75.15 mg of 2-(4-methylpiperazin-1-yl)ethanamine. After spin-filter-drying of the precipitate formed, washing with 3 times 0.5 ml of tetrahydrofuran and twice 0.5 ml of diethyl ether, and air-drying, 0.110 g of 1-[2-(4-methylpiperazin-1-yl)ethyl]-3-[6-([1,2,4]triazolo[4,3-a]pyridin-3-ylsulphanyl)-1,3-benzothiazol-2-yl]urea is obtained in the form of a white solid.

Melting point: 180-185° C. (Köfler bench)

MS: method A; [M+H]+ m/z=469; [M+H—C₈H₁₅N₃O]+ m/z=300 (base peak); [C₈H₁₆N₃O]+ m/z=170; [M−H]− m/z=467; Tr=0.44 min.

¹H NMR (400 MHz, DMSO-d₆) δ ppm 2.15 (s, 3H) 2.21-2.43 (m, 10H) 3.25 (m partially masked, 2H) 6.72 (broad m, 1H) 7.11 (td, J=6.8, 1.0 Hz, 1H) 7.30 (dd, J=8.3, 2.0 Hz, 1H) 7.47-7.54 (m, 2H) 7.90 (dt, J=9.3, 1.0 Hz, 1H) 7.98 (d, J=2.0 Hz, 1H) 8.48 (dt, J=6.8, 1.0 Hz, 1H) 10.91 (broad m, 1H).

### Example 5

### 1-(2-methoxyethyl)-3-[6-([1,2,4]triazolo[4,3-a]pyridin-3-ylsulphanyl)-1,3-benzothiazol-2-yl]urea

The compound can be prepared as in Example 3a, but using 0.2 g of phenyl [[6-([1,2,4]triazolo[4,3-a]pyridin-3-ylsulphanyl)-1,3-benzothiazol-2-yl]-carbamate and 0.05 ml of 2-methoxyethanamine. After spin-filter-drying of the precipitate formed, washing with 3 times 2 ml of diisopropyl ether, and oven-drying at around 50° C. under reduced pressure, 0.143 g of 1-(2-methoxyethyl)-3-[6-([1,2,4]triazolo[4,3-a]pyridin-3-ylsulphanyl)-1,3-benzothiazol-2-yl]urea is obtained in the form of a white solid.

Melting point: 252-257° C. (Köfler bench)

MS: method A; [M+H]+ m/z=401; [M−H]− m/z=399; Tr=0.62 min.

¹H NMR (400 MHz, DMSO-d₆) δ ppm 3.27 (s, 3H) 3.31 (m partially masked, 2H) 3.40 (t, J=5.4 Hz, 2H) 6.83 (broad t, J=5.6 Hz, 1H) 7.11 (td, J=6.8, 1.0 Hz, 1H) 7.31 (dd, J=8.3, 2.0 Hz, 1H) 7.50 (ddd, J=9.3, 6.8, 1.0 Hz, 1H) 7.55 (d, J=8.3 Hz, 1H) 7.91 (dt, J=9.3, 1.0 Hz, 1H) 8.01 (d, J=2.0 Hz, 1H) 8.49 (dt, J=6.8, 1.0 Hz, 1H) 10.73 (broad m, 1H).

### Example 6

### 6-[(6-iodo[1,2,4]triazolo[4,3-a]pyridin-3-yl)sulphanyl]-1,3-benzothiazol-2-amine

### Example 6a

### 6-[(6-iodo[1,2,4]triazolo[4,3-a]pyridin-3-yl)sulphanyl]-1,3-benzothiazol-2-amine

The compound can be obtained as described in Example 1a, using 230 mg of 4-[(6-iodo[1,2,4]triazolo[4,3-a]pyridin-3-yl)sulphanyl]aniline, 13 ml of acetic acid, 0.24 g of potassium thiocyanate and 32 μl of bromine. 0.25 g of 6-[(6-iodo[1,2,4]triazolo[4,3-a]pyridin-3-yl)sulphanyl]-1,3-benzothiazol-2-amine is thus obtained in the form of an orange powder.

Melting point ˜190° C. (Köfler bench).

MS: method B; [M+H]⁺ m/z=426; [M−H]⁻ m/z=424; Tr=2.98 min.

¹H NMR (400 MHz, DMSO-d₆) δ ppm 7.27 (d, J=8.6 Hz, 1H) 7.31 (dd, J=8.6, 2.0 Hz, 1H) 7.61 (broad s, 2H) 7.65 (dd, J=9.5, 1.4 Hz, 1H) 7.73 (d, J=9.5 Hz, 1H) 7.81 (d, J=2.0 Hz, 1H) 8.71 (broad s, 1H).

### Example 6b

### 4-[(6-iodo[1,2,4]triazolo[4,3-a]pyridin-3-yl)sulphanyl]aniline

The compound can be prepared as in Example 1b, using 4.02 g of stannous chloride dihydrate, 60 ml of ethanol and 1.89 g of 6-iodo-3-[(4-nitrophenyl)sulphanyl][1,2,4]triazolo[4,3-a]pyridine and 4.45 ml of a 12N aqueous solution of hydrochloric acid. 0.23 g of 4-[(6-iodo[1,2,4]triazolo[4,3-a]pyridin-3-yl)sulphanyl]aniline is thus obtained in the form of an orangey-brown solid.

MS: method B; [M+H]⁺ m/z=369; Tr=3.06 min.

### Example 6c

### 6-iodo-3-[(4-nitrophenyl)sulphanyl][1,2,4]triazolo[4,3-a]pyridine

The compound can be prepared as in Example 1c, using 1.18 g of 6-iodo-[1,2,4]triazolo[4,3-a]pyridine-3-thiol, 10 ml of dimethyl sulphoxide and 1.21 g of 4-nitrobenzenediazonium tetrafluoroborate. 1.89 g of 6-iodo-3-[(4-nitrophenyl)sulphanyl][1,2,4]triazolo[4,3-a]pyridine are thus obtained in the form of an orange powder.

MS: method B; [M+H]⁺: m/z 399; Tr=3.74 min.

### Example 6d

### 6-iodo-[1,2,4]triazolo[4,3-a]pyridine-3-thiol

The compound can be prepared in the following way:

A solution of 1.37 g of 2-hydrazinyl-5-iodopyridine, 40 ml of tetrahydrofuran and 1.25 g of N,N′-thiocarbonyldiimidazole is brought to reflux for one hour. After cooling, the reaction medium is concentrated by evaporation under reduced pressure and the resulting powder is then stirred under cold conditions in the presence of 25 ml of water. The resulting precipitate is filtered off, washed with twice 10 ml of water, and air-dried. 1.40 g of 6-iodo-[1,2,4]triazolo[4,3-a]pyridine-3-thiol are thus obtained.

Melting point>264° C. (Köfler bench).

MS: method A; [M+H]⁺: m/z 278; [M−H]⁻: m/z 276; Tr=0.57 min.

### Example 6e

### 2-hydrazinyl-5-iodopyridine

The compound can be obtained as described in patent WO 2006/114213, Example 32A, page 40.

### Example 7

### 6-{[6-(4-fluorophenyl)[1,2,4]triazolo[4,3-a]pyridin-3-yl]sulphanyl}-1,3-benzothiazol-2-amine

### Example 7a

### 6-{[6-(4-fluorophenyl)[1,2,4]triazolo[4,3-a]pyridin-3-yl]sulphanyl}-1,3-benzothiazol-2-amine

The compound can be prepared as described in Example 1a, using 0.24 g of 4-{[6-(4-fluorophenyl)[1,2,4]triazolo[4,3-a]pyridin-3-yl]sulphanyl}aniline, 10 ml of acetic acid, 0.28 g of potassium thiocyanate and 37 μl of bromine diluted in 2 ml of glacial acetic acid. 0.14 g of 6-{[6-(4-fluorophenyl)[1,2,4]triazolo[4,3-a]pyridin-3-yl]sulphanyl}-1,3-benzothiazol-2-amine is thus obtained in the form of a pale pink solid.

Melting point: >264° C. (Köfler bench)

MS: method B; [M+H]⁺ m/z=394; [M−H]⁻ m/z=392; Tr=3.47 min.

¹H NMR (400 MHz, DMSO-d₆) δ ppm 7.27 (d, J=8.3 Hz, 1H) 7.31-7.38 (m, 3 H) 7.60 (broad s, 2H) 7.77 (dd, J=8.6, 5.4 Hz, 2H) 7.82 (dd, J=9.8, 1.5 Hz, 1H) 7.87 (d, J=1.7 Hz, 1H) 7.98 (d, J=9.9 Hz, 1H) 8.59 (broad s, 1H).

The compound 6-{[6-(4-fluorophenyl)[1,2,4]triazolo[4,3-a]pyridin-3-yl]sulphanyl}-1,3-benzothiazol-2-amine can also be obtained in the following way:

35 mg of potassium phosphate, 80 mg of 4-fluorophenylboronic acid and 3 mg of tetrakis(triphenylphosphine)palladium are added to a solution of 20 mg of 6-[(6-iodo[1,2,4]triazolo[4,3-a]pyridin-3-yl)sulphanyl]-1,3-benzo-thiazol-2-amine and 1 ml of dimethyl sulphoxide. The reaction medium is heated at 80° C. for 18 hours. 5 mg of tetrakis(triphenylphosphine)palladium are then added and the medium is again brought to 80° C. for 2 days. After cooling of the reaction medium with an ice bath, 15 ml of water are added and the medium is kept stirring under cold conditions for one hour and then for 18 hours at ambient temperature. The aqueous phase is extracted with 3 times 30 ml of ethyl acetate, and the combined organic phases are dried over sodium sulphate, filtered, and concentrated by evaporation under reduced pressure. 20 mg of 6-{[6-(4-fluorophenyl)[1,2,4]triazolo[4,3-a]pyridin-3-yl]sulphanyl}-1,3-benzothiazol-2-amine are thus obtained.

### Example 7b

### 4-{[6-(4-fluorophenyl)[1,2,4]triazolo[4,3-a]pyridin-3-yl]-sulphanyl}aniline

The compound can be prepared as in Example 1b, using 1.88 g of stannous chloride dihydrate, 25 ml of ethanol, 0.61 g of 6-(4-fluorophenyl)-3-[(4-nitrophenyl)sulphanyl][1,2,4]triazolo[4,3-a]pyridine and 2.06 ml of a 10N aqueous solution of hydrochloric acid. 0.24 g of 4-{[6-(4-fluorophenyl)[1,2,4]triazolo[4,3-a]pyridin-3-yl]sulphanyl}aniline is thus obtained in the form of a yellow solid.

Melting point: 217° C. (Köfler bench)

MS: method A; [M+H]⁺: m/z 337 (base peak); [2M+Na]⁺: m/z 695; Tr=0.81 min.

### Example 7c

### 6-(4-fluorophenyl)-3-[(4-nitrophenyl)sulphanyl][1,2,4]triazolo[4,3-a]pyridine

The compound can be prepared as Example 1c, using 0.83 g of 6-(4-fluorophenyl)[1,2,4]triazolo[4,3-a]pyridine-3-thiol, 8 ml of dimethyl sulphoxide and 0.80 g of 4-nitrobenzenediazonium tetrafluoroborate. 0.61 g of 6-(4-fluorophenyl)-3-[(4-nitrophenyl)sulphanyl][1,2,4]triazolo[4,3-a]pyridine is thus obtained in the form of a brown foam.

MS: method A; [M+H]⁺: m/z=367; Tr=0.98 min.

### Example 7d

### 6-(4-fluorophenyl)[1,2,4]triazolo[4,3-a]pyridine-3-thiol

The compound can be prepared in the following way:

A solution of 1.2 g of 5-(4-fluorophenyl)-2-hydrazinylpyridine, 15 ml of carbon disulphide and 50 ml of chloroform is brought to reflux for 18 hours. 15 ml of carbon disulphide are then added and the reaction medium is kept at reflux for 4 hours, then 15 ml of carbon disulphide are added and the reaction medium is kept at reflux for 2 hours, and then 20 ml of carbon disulphide are added and the reaction medium is kept at reflux for 24 hours. The reaction medium is then kept stirring at ambient temperature for 24 hours. After the addition of 20 ml of ethanol, the medium is brought to reflux for 29 hours.

After cooling, the medium is concentrated by evaporation under reduced pressure and the resulting yellow powder is purified by chromatography, under an argon pressure, on silica gel (eluent: 97/3 dichloromethane/methanol). 0.63 g of 6-(4-fluorophenyl)[1,2,4]triazolo[4,3-a]pyridine-3-thiol is thus obtained in the form of a yellow powder.

Melting point: 249° C. (Köfler bench)

MS: method A; [M+H]⁺: m/z=246; [M−H]⁻: m/z=244; Tr=0.77 min.

### Example 7e

### 5-(4-fluorophenyl)-2-hydrazinylpyridine

The compound can be prepared as described by R. Church et al., Journal of Organic Chemistry (1995), 60(12), 3750-8.

### Example 8

### N-{6-[6-(4-fluorophenyl)[1,2,4]triazolo[4,3-a]pyridin-3-ylsulphanyl]-1,3-benzothiazol-2-yl}cyclopropanecarboxamide

The compound can be prepared as in Example 2, using 0.13 g of 6-{[6-(4-fluorophenyl)[1,2,4]triazolo[4,3-a]pyridin-3-yl]sulphanyl}-1,3-benzothiazol-2-amine, 0.081 ml of cyclopropanecarbonyl chloride and 5 ml of pyridine. 0.11 g of N-{6-[6-(4-fluorophenyl)[1,2,4]triazolo[4,3-a]pyridin-3-ylsulphanyl]-1,3-benzothiazol-2-yl}cyclopropanecarboxamide is thus obtained in the form of a yellow solid.

MS: method B; [M+H]⁺ m/z=462; [M−H]⁻ m/z=460; Tr=0.97 min.

¹H NMR (400 MHz, DMSO-d₆) δ ppm 0.92 (m, 4H) 1.96 (m, 1H) 7.34 (t, J=8.8 Hz, 2H) 7.45 (dd, J=8.4, 2.0 Hz, 1H) 7.66 (d, J=8.4 Hz, 1H) 7.77 (dd, J=8.8, 5.5 Hz, 2H) 7.84 (dd, J=9.6, 1.7 Hz, 1H) 8.01 (dd, J=9.6, 1.0 Hz, 1H) 8.11 (d, J=2.0 Hz, 1H) 8.61 (broad s, 1H) 12.57 (broad m, 1H).

### Example 9

### 6-{[6-(1-methyl-1H-pyrazol-4-yl)[1,2,4]triazolo[4,3-a]pyridin-3-yl]sulphanyl}-1,3-benzothiazol-2-amine

The compound can be prepared in the following way:

A solution of 0.25 g of 6-[(6-iodo[1,2,4]triazolo[4,3-a]pyridin-3-yl)sulphanyl]-1,3-benzothiazol-2-amine, 5 ml of 1-2-dimethoxyethane, 1.2 ml of NaOH (1N aqueous solution) and 0.14 g of (1-methyl-1H-pyrazol-4-yl)boronic acid is stirred under argon for 30 minutes. 20 mg of dichlorobis(triphenylphosphine)-palladium are then added and the reaction medium is brought to 65° C. for 30 minutes. 20 mg of dichlorobis(triphenylphosphine)palladium are then added and the reaction medium is brought to reflux overnight. A further 20 mg of dichlorobis(triphenylphosphine)palladium and 0.61 g of (1-methyl-1H-pyrazol-4-yl)boronic acid are added. The medium is brought to reflux for 4 hours and left to stir at a temperature in the region of 20° C. for 2 days. 10 ml of dioxane, 1 ml of water and 20 mg of dichlorobis(triphenylphosphine)-palladium are then added and the medium is transferred into a sealed tube and brought to 150° C., with microwaves, for 15 minutes. After a return to a temperature in the region of 20° C., the medium is concentrated by evaporation under reduced pressure. The residue thus obtained is chromatographed, under an argon pressure, on silica gel (eluent: 95/5 dichloromethane/-methanol). 0.14 g of 6-{[6-(1-methyl-1H-pyrazol-4-yl)[1,2,4]triazolo[4,3-a]pyridin-3-yl]sulphanyl}-1,3-benzothiazol-2-amine is thus obtained in the form of an orangey-brown solid.

MS: method A; [M+H]⁺ m/z=380; [M−H]⁻ m/z=378; Tr=0.5 min.

¹H NMR (400 MHz, DMSO-d₆) δ ppm 3.88 (s, 3H) 7.27 (d, J=8.3 Hz, 1H) 7.35 (dd, J=8.3, 2.0 Hz, 1H) 7.60 (s, 2H) 7.75 (dd, J=9.5, 1.3 Hz, 1H) 7.87 (d, J=2 Hz, 1H) 7.91 (d, J=9.5 Hz, 1H) 8.02 (s, 1H) 8.33 (s, 1H) 8.57 (s, 1H).

### Example 10

### N-(6-{[6-(1-methyl-1H-pyrazol-4-yl)[1,2,4]triazolo[4,3-a]pyridin-3-yl]sulphanyl}-1,3-benzothiazol-2-yl)cyclopropane-carboxamide

The compound can be prepared as in Example 2, using 0.13 g of 6-{[6-(1-methyl-1H-pyrazol-4-yl)[1,2,4]triazolo[4,3-a]pyridin-3-yl]sulphanyl}-1,3-benzothiazol-2-amine, 0.034 ml of cyclopropanecarbonyl chloride and 2 ml of pyridine. 0.1 g of N-(6-{[6-(1-methyl-1H-pyrazol-4-yl)[1,2,4]triazolo[4,3-a]pyridin-3-yl]sulphanyl}-1,3-benzothiazol-2-yl)cyclopropanecarboxamide is thus obtained in the form of a pale yellow solid.

Melting point ˜196° C. (Köfler bench).

MS: method B; [M+H]⁺ m/z=448; [M−H]⁻ m/z=446; Tr=3.32 min.

¹H NMR (400 MHz, DMSO-d₆) δ ppm 0.89-1.00 (m, 4H) 1.94-2.01 (m, 1H) 3.87 (s, 3H) 7.43 (dd, J=8.5, 2.0 Hz, 1H) 7.67 (d, J=8.5 Hz, 1H) 7.77 (dd, J=9.5, 1.5 Hz, 1H) 7.94 (d, J=9.5 Hz, 1H) 8.01 (s, 1H) 8.12 (d, J=2 Hz, 1H) 8.33 (s, 1H) 8.58 (s, 1H) 12.62 (br. s., 1H).

### Example 11

### N-(6-{[6-(1H-pyrazol-4-yl)[1,2,4]triazolo[4,3-a]pyridin-3-yl]-sulphanyl}-1,3-benzothiazol-2-yl)cyclopropanecarboxamide

### Example 11a

### N-(6-{[6-(1H-pyrazol-4-yl)[1,2,4]triazolo[4,3-a]pyridin-3-yl]-sulphanyl}-1,3-benzothiazol-2-yl)cyclopropanecarboxamide

The compound can be prepared in the following way:

104 mg of 3-bromo-6-(1H-pyrazol-4-yl)[1,2,4]triazolo[4,3-a]pyridine, 100 mg of (6-sulphanyl-1,3-benzothiazol-2-yl)cyclopropanecarboxamide, 110 mg of potassium carbonate and 1 ml of dimethyl sulphoxide are charged to a sealed glass tube. The medium is microwave-heated at 185° C. for 12 minutes. After a return to a temperature in the region of 20° C., the medium is poured into 60 ml of water and the precipitate thus formed is filtered off through sintered glass, washed with water, spin-filter-dried and dried. The solid thus obtained is chromatographed, under an argon pressure, on silica gel (eluent: 85/15 then 90/10 dichloromethane/methanol). A solid is thus obtained, and is triturated from 2 ml of ethanol, filtered, washed twice with 1 ml of ethanol and then 3 times with 1 ml of diethyl ether, and dried. 82 mg of N-(6-{[6-(1H-pyrazol-4-yl)[1,2,4]triazolo[4,3-a]pyridin-3-yl]sulphanyl}-1,3-benzothiazol-2-yl)cyclo-propanecarboxamide are thus obtained in the form of a pale yellow solid.

Melting point>260° C. (Köfler bench).

MS: method A; [M+H]⁺ m/z=434; Tr=0.65 min.

¹H NMR (400 MHz, DMSO-d₆) δ ppm 0.89-0.99 (m, 4H) 1.94-2.03 (m, 1H) 7.45 (dd, J=8.5, 2.0 Hz, 1H) 7.67 (d, J=8.5 Hz, 1H) 7.83 (dd, J=9.5, 1.7 Hz, 1H) 7.94 (dd, J=9.5, 1.0 Hz, 1H) 8.08 (br. s., 1H) 8.15 (d, J=2.0 Hz, 1H) 8.39 (br. s., 1H) 8.59-8.65 (m, 1H) 12.66 (br. s., 1H) 13.11 (br. s., 1H).

### Example 11b

### (6-sulphanyl-1,3-benzothiazol-2-yl)cyclopropanecarboxamide

The compound can be prepared in the following way:

A solution of 33.6 mg of potassium dihydrogen phosphate in 8 ml of water to 20° C., is added to a suspension of 2 g of (6-thiocyanato-1,3-benzothiazol-2-yl)cyclopropanecarboxamide and 70 ml of ethanol, followed by 3.2 g of DL-dithiothreitol. The reaction medium is stirred at reflux for 5 h and then brought to a temperature in the region of 20° C. 400 ml of water are then added and the precipitate formed is filtered off through sintered glass, washed thoroughly with water, spin-filter-dried, and then dried. 1.5 g of (6-sulphanyl-1,3-benzothiazol-2-yl)cyclopropanecarboxamide are thus obtained in the form of a pale yellow solid.

MS: method B; [M+H]⁺ m/z=251; [M−H]⁻ m/z=249; Tr=3.77 min.

### Example 11c

### (6-thiocyanato-1,3-benzothiazol-2-yl)cyclopropanecarboxamide

The compound can be prepared in the following way:

5.3 ml of cyclopropanecarbonyl chloride are added to a solution of 10 g of 2-amino-1,3-benzothiazol-6-yl thiocyanate (commercial product) and 100 ml of pyridine, while maintaining the temperature in the region of 20° C. The reaction medium is stirred for 4 hours and then 500 ml of water are added. The precipitate formed is filtered off through sintered glass, washed thoroughly with water, spin-filter-dried, and then dried. 13 g of (6-thiocyanato-1,3-benzothiazol-2-yl)cyclopropanecarboxamide are thus obtained in the form of a pale yellow solid, said compound being used as it is in the subsequent stages.

### Example 11d

### 3-bromo-6-(1H-pyrazol-4-yl)[1,2,4]triazolo[4,3-a]pyridine

The compound can be prepared in the following way:

A solution of 0.058 ml of bromine and 2 ml of water is added to a solution of 170 mg of 6-(1H-pyrazol-4-yl)[1,2,4]triazolo[4,3-a]pyridine in 4 ml of ethanol. The reaction mixture is stirred for approximately 2 days at a temperature in the region of 20° C., and then 20 ml of a saturated aqueous solution of sodium hydrogen carbonate are added. After stirring for 30 minutes, the precipitate formed is filtered off through sintered glass, washed with three times 5 ml of water, spin-filter-dried, and then dried. The solid residue obtained is chromatographed, under an argon pressure, on silica gel (eluent: 85/15 ethyl acetate/methanol). 110 mg of 3-bromo-6-(1H-pyrazol-4-yl)[1,2,4]triazolo[4,3-a]pyridine are thus obtained in the form of a white solid.

MS: method A; [M+H]⁺ m/z=264; [M−H]⁻ m/z=262; Tr=0.35 min.

### Example 11e

### 6-(1H-pyrazol-4-yl)[1,2,4]triazolo[4,3-a]pyridine

The compound can be prepared in the following way:

272 mg of (1H-pyrazol-4-yl)boronic acid are added to a mixture of 400 mg of 6-bromo[1,2,4]triazolo[4,3-a]pyridine (commercial product), 8 ml of dimethyl sulphoxide, 69 mg of tetrakis(triphenylphosphine)palladium and 424 mg of sodium carbonate in solution in 2 ml of water. The reaction medium is microwave heated at 150° C. for 20 minutes. After a return to a temperature in the region of 20° C., the medium is concentrated by evaporation under reduced pressure, and then taken up with 40 ml of water. The aqueous phase is extracted with 3 times 20 ml of ethyl acetate. The precipitate formed in the aqueous phase is filtered off through sintered glass, washed with water, spin-filter-dried, and then dried. 200 mg of 6-(1H-pyrazol-4-yl)[1,2,4]triazolo[4,3-a]-pyridine are thus obtained in the form of a white solid.

MS: method A; [M+H]⁺ m/z=186; [M−H]⁻ m/z=184; Tr=0.21 min.

### Example 12

### N-(6-{[6-((3-fluoro-4-methyl)phenyl)[1,2,4]triazolo[4,3-a]pyridin-3-yl]sulphanyl}-1,3-benzothiazol-2-yl)cyclopropanecarboxamide

### Example 12a

### N-(6-{[6-((3-fluoro-4-methyl)phenyl)[1,2,4]triazolo[4,3-a]pyridin-3-yl]sulphanyl}-1,3-benzothiazol-2-yl)cyclopropanecarboxamide

The compound can be prepared as in Example 11a, using 348 mg of 3-bromo-6-((3-fluoro-4-methyl)phenyl)[1,2,4]triazolo[4,3-a]pyridine, 250 mg of (6-sulphanyl-1,3-benzothiazol-2-yl)cyclopropanecarboxamide, 280 mg of potassium carbonate and 4 ml of dimethyl sulphoxide. 146 mg of N-(6-{[6-((3-fluoro-4-methyl)phenyl)[1,2,4]triazolo[4,3-a]pyridin-3-yl]sulphanyl}-1,3-benzothiazol-2-yl)cyclopropanecarboxamide are thus obtained in the form of a white solid.

Melting point=191° C. (Köfler bench).

MS: method A; [M+H]⁺ m/z=476; [M−H]⁻ m/z=474; Tr=1.04 min.

¹H NMR (400 MHz, DMSO-d₆) δ ppm 0.89-0.96 (m, 4H) 1.93-2.00 (m, 1H) 2.28 (d, J=1.5 Hz, 3H) 7.40-7.49 (m, 3H) 7.57 (dd, J=11.2, 1.5 Hz, 1H) 7.67 (d, J=8.5 Hz, 1H) 7.87 (dd, J=9.5, 1.5 Hz, 1H) 7.99 (dd, J=9.5, 1.5 Hz, 1H) 8.13 (d, J=1.7 Hz, 1H) 8.61-8.66 (m, 1H) 12.65 (br. s., 1H).

### Example 12b

### 3-bromo-6-((3-fluoro-4-methyl)phenyl)[1,2,4]triazolo[4,3-a]-pyridine

The compound can be prepared in the following way:

A mixture of 450 mg of 6-((3-fluoro-4-methyl)phenyl)[1,2,4]triazolo[4,3-a]pyridine, 10 ml of chloroform and 356 mg of N-bromosuccinimide is brought to reflux overnight. The medium is cooled to a temperature in the region of 20° C. and then concentrated by evaporation under reduced pressure. The residue thus obtained is chromatographed, under an argon pressure of silica gel (eluent: 80/20 ethyl acetate/methanol). 534 mg of 3-bromo-6-((3-fluoro-4-methyl)phenyl)[1,2,4]triazolo[4,3-a]pyridine are thus obtained in the form of a beige solid.

MS: method A; [M+H]⁺ m/z=306; Tr=0.88 min.

### Example 12c

### 6-((3-fluoro-4-methyl)phenyl)[1,2,4]triazolo[4,3-a]pyridine

The compound can be prepared as in Example 11e, using 400 mg of 6-bromo[1,2,4]triazolo[4,3-a]pyridine (commercial product), 8 ml of dimethyl sulphoxide, 69 mg of tetrakis(triphenylphosphine)palladium, 424 mg of sodium carbonate in solution in 2 ml of water and 370 mg of ((3-fluoro-4-methyl)phenyl)boronic acid. 456 mg of 6-((3-fluoro-4-methyl)phenyl)[1,2,4]-triazolo[4,3-a]pyridine are thus obtained in the form of a white solid.

Melting point=236° C. (Köfler bench).

MS: method A; [M+H]⁺ m/z=228; Tr=0.71 min.

### Example 13

### N-(6-{[6-(3-fluorophenyl)[1,2,4]triazolo[4,3-a]pyridin-3-yl]sulphanyl}-1,3-benzothiazol-2-yl)cyclopropanecarboxamide

### Example 13a

### N-(6-{[6-(3-fluorophenyl)[1,2,4]triazolo[4,3-a]pyridin-3-yl]sulphanyl}-1,3-benzothiazol-2-yl)cyclopropanecarboxamide

The compound can be prepared as in Example 11a using 480 mg of 3-bromo-6-(3-fluorophenyl)[1,2,4]triazolo[4,3-a]pyridine, 411 mg of (6-sulphanyl-1,3-benzothiazol-2-yl)cyclopropanecarboxamide, 454 mg of potassium carbonate and 10 ml of dimethyl sulphoxide. 148 mg of N-(6-{[6-(3-fluorophenyl)[1,2,4]triazolo[4,3-a]pyridin-3-yl]sulphanyl}-1,3-benzothiazol-2-yl)cyclopropanecarboxamide are thus obtained in the form of a beige solid.

Melting point>260° C. (Köfler bench).

MS: method A; [M+H]⁺ m/z=462; [M−H]⁻ m/z=460; Tr=0.98 min.

¹H NMR (400 MHz, DMSO-d₆) δ ppm 0.92 (br. s., 4H) 1.95 (br. s., 1H) 7.23-7.31 (m, 1H) 7.46 (d, J=8.6 Hz, 1H) 7.50-7.70 (m, 4H) 7.88 (dd, J=9.5, 1.5 Hz, 1H) 8.01 (dd, J=9.5, 1.5 Hz, 1H) 8.13 (br. s., 1H) 8.69 (br. s., 1H).

### Example 13b

### 3-bromo-6-(3-fluorophenyl)[1,2,4]triazolo[4,3-a]pyridine

The compound can be prepared as in Example 12b, using 360 mg of 6-(3-fluorophenyl)[1,2,4]triazolo[4,3-a]pyridine, 10 ml of chloroform and 300 mg of N-bromosuccinimide. 480 mg of 3-bromo-6-(3-fluorophenyl)[1,2,4]triazolo[4,3-a]pyridine are thus obtained in the form of an ochre solid.

MS: method A; [M+H]⁺ m/z=292; Tr=0.77 min.

### Example 13c

### 6-(3-fluorophenyl)[1,2,4]triazolo[4,3-a]pyridine

The compound can be prepared as in Example 12c, using 400 mg of 6-bromo[1,2,4]triazolo[4,3-a]pyridine (commercial product), 8 ml of dimethyl sulphoxide, 69 mg of tetrakis(triphenylphosphine)palladium, 424 mg of sodium carbonate in solution in 2 ml of water and 345 mg of (3-fluoro-phenyl)boronic acid. 361 mg of 6-((3-fluoro-4-methyl)phenyl)[1,2,4]-triazolo[4,3-a]pyridine are thus obtained in the form of a white solid.

Melting point=210° C. (Köfler bench).

MS: method A; [M+H]⁺ m/z=214; Tr=0.59 min.

### Example 14

### N-(6-{[6-(1-[2-(tetrahydro-2H-pyran-2-yloxy)ethyl]-1H-pyrazol-4-yl)[1,2,4]triazolo[4,3-a]pyridin-3-yl]sulphanyl}-1,3-benzo-thiazol-2-yl)cyclopropanecarboxamide

### Example 14a

### N-(6-{[6-(1-[2-(tetrahydro-2H-pyran-2-yloxy)ethyl]-1H-pyrazol-4-yl)[1,2,4]triazolo[4,3-a]pyridin-3-yl]sulphanyl}-1,3-benzothiazol-2-yl)cyclo-propanecarboxamide

The compound can be prepared as in Example 11a, using 240 mg of 3-bromo-6-(1-[2-(tetrahydro-2H-pyran-2-yloxy)ethyl]-1H-pyrazol-4-yl)[1,2,4]-triazolo[4,3-a]pyridine, 170 mg of (6-sulphanyl-1,3-benzothiazol-2-yl)cyclo-propanecarboxamide, 170 mg of potassium carbonate and 4 ml of dimethyl sulphoxide. 240 mg of N-(6-{[6-(1-[2-(tetrahydro-2H-pyran-2-yloxy)ethyl]-1H-pyrazol-4-yl)[1,2,4]triazolo[4,3-a]pyridin-3-yl]sulphanyl}-1,3-benzothiazol-2-yl)cyclopropanecarboxamide are thus obtained in the form of a white solid.

Melting point ˜110° C. (Köfler bench).

MS: method A; [M+H]⁺ m/z=562; [M−H]⁻ m/z=560; Tr=0.84 min.

¹H NMR (400 MHz, DMSO-d₆) δ ppm 0.90-0.98 (m, 4H) 1.27-1.67 (m, 6H) 1.91-2.01 (m, 1H) 3.32-3.39 (m, 1H) 3.52 (ddd, J=11.5, 8.6, 3.4 Hz, 1H) 3.70-3.80 (m, 1H) 3.89-3.98 (m, 1H) 4.23-4.36 (m, 2H) 4.51 (t, J=3.3 Hz, 1H) 7.42 (dd, J=8.6, 2.0 Hz, 1H) 7.66 (d, J=8.6 Hz, 1H) 7.78 (dd, J=9.5, 1.5 Hz, 1H) 7.94 (dd, J=9.5, 1.0 Hz, 1H) 8.05 (s, 1H) 8.11 (d, J=2.0 Hz, 1H) 8.36 (s, 1H) 8.58 (s, 1H) 12.65 (br. s., 1H).

### Example 14b

### 3-bromo-6-(1-[2-(tetrahydro-2H-pyran-2-yloxy)ethyl]-1H-pyrazol-4-yl)[1,2,4]triazolo[4,3-a]pyridine

The compound can be prepared as in Example 12b, using 440 mg of 6-(1-[2-(tetrahydro-2H-pyran-2-yloxy)ethyl]-1H-pyrazol-4-yl)[1,2,4]triazolo[4,3-a]-pyridine, 10 ml of chloroform and 226 mg of N-bromosuccinimide. 245 mg of 3-bromo-6-(1-[2-(tetrahydro-2H-pyran-2-yloxy)ethyl]-1H-pyrazol-4-yl)[1,2,4]triazolo[4,3-a]pyridine are thus obtained in the form of a colourless lacquer.

MS: method A; [M+H]⁺ m/z=392; Tr=0.64 min.

### Example 14c

### 6-(1-[2-(tetrahydro-2H-pyran-2-yloxy)ethyl]-1H-pyrazol-4-yl)[1,2,4]triazolo[4,3-a]pyridine

The compound can be prepared as in Example 9, using 320 mg of 6-bromo-[1,2,4]triazolo[4,3-a]pyridine (commercial product), 15 ml of 1,2-dimethoxyethane, 69 mg of dichlorobis(triphenylphosphine)palladium, 3.2 ml of NaOH (1N aqueous solution) and 990 mg of 1-[2-(tetrahydro-2H-pyran-2-yloxy)ethyl]-4-(4,4,5,5-tetramethyl-1,3,2-dioxaborolan-2-yl)-1H-pyrazole. 445 mg of 6-(1-[2-(tetrahydro-2H-pyran-2-yloxy)ethyl]-1H-pyrazol-4-yl)[1,2,4]-triazolo[4,3-a]pyridine are thus obtained in the form of a yellow oil which crystallizes.

MS: method A; [M+H]⁺ m/z=314; Tr=0.49 min.

### Example 14d

### 1-[2-(tetrahydro-2H-pyran-2-yloxy)ethyl]-4-(4,4,5,5-tetramethyl-1,3,2-dioxaborolan-2-yl)-1H-pyrazole

The compound can be prepared as described in patent US2007/0265272, page 39.

### Example 15

### N-(6-{[6-(1-(2-hydroxyethyl)-1H-pyrazol-4-yl)[1,2,4]triazolo-[4,3-a]pyridin-3-yl]sulphanyl}-1,3-benzothiazol-2-yl)cyclopropane-carboxamide

The compound can be prepared in the following way:

45 mg of Amberlyst 15 form H+ resin are added to a solution of 215 mg of N-(6-{[6-(1-[2-(tetrahydro-2H-pyran-2-yloxy)ethyl]-1H-pyrazol-4-yl)[1,2,4]-triazolo[4,3-a]pyridin-3-yl]sulphanyl}-1,3-benzothiazol-2-yl)cyclopropane-carboxamide and 10 ml of methanol, and the reaction medium is stirred for 16 h at a temperature in the region of 20° C. After the addition of 5 ml of dichloromethane, resin is again added so as to complete the reaction (monitored by LC/MS), i.e. successively 45 mg, 40 mg, then 150 mg of resin, while at the same stirring at a temperature in the region of 20° C. and over a total period of 4 days. The reaction medium is then filtered and the resin is washed with 4 times 15 ml of a mixture of CH₂Cl₂/MeOH/NH₄OH at 28% (12/3/0.5 by volume). The filtrate obtained is concentrated by evaporation under reduced pressure. 65 mg of N-(6-{[6-(1-(2-hydroxyethyl)-1H-pyrazol-4-yl)[1,2,4]triazolo[4,3-a]pyridin-3-yl]sulphanyl}-1,3-benzothiazol-2-yl)cyclopropanecarboxamide are thus obtained in the form of a white solid.

Melting point ˜182° C. (Köfler bench).

MS: method A; [M+H]⁺ m/z=478; [M−H]⁻ m/z=476; Tr=0.63 min.

¹H NMR (400 MHz, DMSO-d₆) δ ppm 0.88-1.00 (m, 4H) 1.91-2.03 (m, 1 H) 3.75 (q, J=5.5 Hz, 2H) 4.16 (t, J=5.6 Hz, 2H) 4.95 (t, J=5.3 Hz, 1H) 7.44 (dd, J=8.6, 2.0 Hz, 1H) 7.67 (d, J=8.3 Hz, 1H) 7.81 (dd, J=9.5, 1.5 Hz, 1H) 7.95 (d, J=9.3 Hz, 1H) 8.06 (s, 1H) 8.12 (d, J=2.0 Hz, 1H) 8.38 (s, 1H) 8.62 (s, 1H).

### Example 16

### N-(6-{[6-(1-piperidin-4-yl-1H-pyrazol-4-yl) [1,2,4]triazolo[4,3-a]pyridin-3-yl]sulphanyl}-1,3-benzothiazol-2-yl)cyclopropanecarboxamide

### Example 16a

### N-(6-{[6-(1-piperidin-4-yl-1H-pyrazol-4-yl)[1,2,4]triazolo[4,3-a]pyridin-3-yl]sulphanyl}-1,3-benzothiazol-2-yl)cyclopropanecarboxamide

The compound can be prepared in the following way:

A mixture of 102 mg of 2-methylpropan-2-yl 4-{4-[3-({2-[(cyclopropylcarbonyl)amino]-1,3-benzothiazol-6-yl}sulphanyl)[1,2,4]triazolo-[4,3-a]pyridin-6-yl]-1H-pyrazol-1-yl}piperidine-1-carboxylate and 1.52 ml of hydrochloric acid (4N solution in dioxane) is stirred at a temperature in the region of 20° C. overnight, and then concentrated by evaporation under reduced pressure. The residue thus obtained is taken up with 5 ml of diisopropyl ether and then filtered through sintered glass, washed with twice 2 ml of diisopropyl ether, spin-filter-dried, and then dried. 101 mg of N-(6-{[6-(1-piperidin-4-yl-1H-pyrazol-4-yl)[1,2,4]triazolo[4,3-a]pyridin-3-yl]sulphanyl}-1,3-benzothiazol-2-yl)cyclopropanecarboxamide hydrochloride are obtained in the form of an ochre solid.

Melting point>260° C. (Köfler bench).

MS: method B; [M+H]⁺ m/z=517; [M−H]⁻ m/z=515; Tr=2.66 min.

¹H NMR (400 MHz, DMSO-d₆) δ ppm 0.85-1.01 (m, 4H) 1.93-2.05 (m, 1 H) 2.05-2.30 (m, 4H) 3.02-3.18 (m, 2H) 3.33-3.44 (m, 2H) 4.42-4.57 (m, 1H) 7.44 (dd, J=8.7, 1.6 Hz, 1H) 7.68 (d, J=8.6 Hz, 1H) 7.86 (d, J=9.5 Hz, 1H) 7.98 (d, J=9.8 Hz, 1H) 8.13 (s, 2H) 8.48 (s, 1H) 8.67 (s, 1H) 12.70 (s, 1H).

### Example 16b

### 2-methylpropan-2-yl 4-{4-[3-({2-[(cyclopropylcarbonyl)amino]-1,3-benzothiazol-6-yl}sulphanyl)[1,2,4]triazolo[4,3-a]pyridin-6-yl]-1H-pyrazol-1-yl}piperidine-1-carboxylate

The compound can be prepared as in Example 11a, using 134 mg of 2-methylpropan-2-yl 4-{4-[(3-bromo[1,2,4]triazolo[4,3-a]pyridin)-6-yl]-1H-pyrazol-1-yl}piperidine-1-carboxylate, 83 mg of (6-sulphanyl-1,3-benzothiazol-2-yl)cyclopropanecarboxamide, 83 mg of potassium carbonate and 3.5 ml of dimethyl sulphoxide. 103 mg of 2-methylpropan-2-yl 4-{-[3-({2-[(cyclopropylcarbonyl)amino]-1,3-benzothiazol-6-yl}sulphanyl)[1,2,4]triazolo[4,3-a]pyridin-6-yl]-1H-pyrazol-1-yl}piperidine-1-carboxylate are thus obtained in the form of a beige solid.

MS: method A; [M+H]⁺ m/z=617; [M−H]⁻ m/z=615; Tr=0.99 min.

### Example 16c

### 2-methylpropan-2-yl 4-{4-[(3-bromo[1,2,4]triazolo[4,3-a]pyridin)-6-yl]-1H-pyrazol-1-yl}piperidine-1-carboxylate

The compound can be prepared as in Example 12b, using 120 mg of 2-methylpropan-2-yl 4-[4-([1,2,4]triazolo[4,3-a]pyridin-6-yl)-1H-pyrazol-1-yl]piperidine-1-carboxylate, 5 ml of chloroform and 58 mg of N-bromosuccinimide. 134 mg of 2-methylpropan-2-yl 4-{4-[(3-bromo[1,2,4]triazolo[4,3-a]pyridin)-6-yl]-1H-pyrazol-1-yl}piperidine-1-carboxylate are thus obtained in the form of a green solid.

MS: method B; [M+H]⁺ m/z=447; [M−H]⁻+HCOOH m/z=491; Tr=3.71 min.

### Example 16d

### 2-methylpropan-2-yl 4-[4-([1,2,4]triazolo[4,3-a]pyridin-6-yl)-1H-pyrazol-1-yl]piperidine-1-carboxylate

The compound can be prepared as in Example 9, using 180 mg of 6-bromo-[1,2,4]triazolo[4,3-a]pyridine (commercial product), 10 ml of 1,2-dimethoxyethane, 35 mg of dichlorobis(triphenylphosphine)palladium, 1.8 ml of NaOH (1N aqueous solution) and 377 mg of tert-butyl 4-[4-(4,4,5,5-tetramethyl-1,3,2-dioxaborolan-2-yl)pyrazol-1-yl]piperidine-1-carboxylate. 120 mg of 2-methylpropan-2-yl 4-[4-([1,2,4]triazolo[4,3-a]pyridin-6-yl)-1H-pyrazol-1-yl]piperidine-1-carboxylate are thus obtained in the form of a colourless lacquer.

MS: method B; [M+H]⁺ m/z=369; [M−H]⁻+HCOOH m/z=413; Tr=3.25 min.

### Example 16e

### tert-butyl 4-[4-(4,4,5,5-tetramethyl-1,3,2-dioxaborolan-2-yl)pyrazol-1-yl]piperidine-1-carboxylate

The compound can be prepared as described in patent WO2007/066187, page 34.

### Example 17

### Pharmaceutical Composition

Tablets corresponding to the following formula were prepared:

### Product of Example 7 . . . 0.2 g

Excipient for a finished tablet of . . . 1 g
(excipient details: lactose, talc, starch, magnesium stearate).

Example 7 is taken as an example of a pharmaceutical preparation, it being possible for this preparation to be carried out, if desired, with other products in the examples in the present invention.

### Pharmacological Section:

### Experimental Protocols

### I) Expression and Purification of Met, Cytoplasmic Domain

### Expression in Baculovirus:

The His-Tev-MET (956-1390) recombinant DNA in pFastBac (Invitrogen) is transfected into insect cells and, after several viral amplification steps, the final baculovirus stock is tested for the expression of the protein of interest.

After infection for 72 h at 27° C. with the recombinant virus, SF21 cell cultures are harvested by centrifugation and the cell pellets are stored at −80° C.

### Purification:

The cell pellets are resuspended in the lysis buffer (buffer A [50 mM HEPES, pH 7.5, 250 mM NaCl, 10% glycerol, 1 mM TECP]; +cocktail of protease inhibitors, Roche Diagnostics, without EDTA, ref 1873580), stirred at 4° C. until the mixture is homogeneous, and then lysed mechanically using a “Dounce” type apparatus.

After centrifugation, the lysis supernatant is incubated for 2 h at 4° C. with nickel chelate resin (His-Trap 6 Fast Flow™, GE HealthCare). After washing with 20 volumes of buffer A, the suspension is packed into a column, and the proteins are eluted with a gradient of buffer B (buffer A+290 mM imidazole).

The fractions containing the protein of interest, for the purpose of electrophoretic analysis (SDS PAGE), are combined, concentrated by ultrafiltration (10 kDa cut-off) and injected onto an exclusion chromatography column (Superdex™ 200, GE HealthCare) equilibrated in buffer A.

After enzymatic cleavage of the histidine tag, the protein is reinjected onto a new IMAC nickel chelate chromatography column (His-Trap 6 Fast Flow™, GE HealthCare) equilibrated in buffer A. The fractions eluted with a gradient of buffer B and containing the protein of interest after electrophoresis (SDS PAGE) are finally combined and stored at −80° C.

For the production of autophosphorylated protein, the previous fractions are incubated for 1 h at ambient temperature after the addition of 2 mM ATP, 2 mM MgCl₂ and 4 mM Na₃VO₄. After the reaction has been stopped with 5 mM of EDTA, the reaction mixture is injected onto a HiPrep desalifying column (GE HealthCare) pre-equilibrated in buffer A+4 mM Na₃VO₄, and the fractions containing the protein of interest (SDS PAGE analysis) are combined and stored at −80° C. The degree of phosphorylation is verified by mass spectrometry (LC-MS) and by peptide mapping.

### II) Tests A and B

### A) Test A: HTRF MET Assay in 96-Well Format

MET at a final concentration of 5 nM is incubated in a final volume of 50 μl of enzymatic reaction in the presence of the test molecule (for a final concentration range of from 0.17 nM to 10 μM, 3% DMSO final concentration) in 10 mM MOPS buffer, pH 7.4, 1 mM DTT, 0.01% Tween 20. The reaction is initiated with the substrate solution to obtain final concentrations of 1 μg/ml poly-(GAT), 10 μM ATP and 5 mM MgCl₂. After incubation for 10 min at ambient temperature, the reaction is stopped with a 30 μl mix so as to obtain a final solution of 50 mM Hepes, pH 7.5, 500 mM potassium fluoride, 0.1% BSA and 133 mM EDTA in the presence of 80 ng of streptavidin 61SAXLB Cis-Bio Int. and 18 ng of anti-phosphotyrosine Mab PT66-Europium Cryptate per well. After incubation for 2 hours at ambient temperature, the reading is taken at 2 wavelengths, 620 nm and 665 nm, on a reader for the TRACE/HTRF technique, and the % inhibition is calculated from the 665/620 ratios.

The results obtained for this test A for the products of formula (I) in examples in the experimental section are such that the IC₅₀ is less than 500 nM, and in particular less than 100 nM.

B) Test B: Inhibition of the autophosphorylation of MET; ELISA technique (pppY1230,1234,1235)
a) Cell lysates: MKN45 cells are seeded into 96-well plates (cell coat BD polylysine) at 20 000 cells/well in 200 μl in RPMI medium+10% FCS+1% L-glutamine. They are left to adhere for 24 hours in an incubator.

The cells are treated the day after seeding with the products at 6 concentrations, in duplicate, for 1 h. At least 3 control wells are treated with the same final amount of DMSO.

Product dilution: Stock at 10 mM in pure DMSO—range from 10 mM to 30 μM with an increment of 3 in pure DMSO—intermediate dilutions to 1/50 in culture medium and then removal of 10 μl added directly to the cells (200 μl): final range of 10 000 to 30 nM.

At the end of the incubation, the supernatant is carefully removed and rinsing is performed with 200 μl of PBS. Next, 100 μl of lysis buffer are placed directly in the wells on ice and incubated at 4° C. for 30 minutes. Lysis buffer: 10 mM Tris HCl, pH 7.4, 100 mM NaCl, 1 mM EDTA, 1 mM EGTA, 1% Triton X-100, 10% glycerol, 0.1% SDS, 0.5% deoxycholate, 20 mM NaF, 2 mM Na₃VO₄, 1 mM PMSF and cocktail of antiproteases.

The 100 μl of lysates are transferred into a V-bottomed polypropylene plate and the ELISA is performed immediately, or the plate is frozen at −80° C.

### b) PhosphoMET ELISA BioSource kit KHO0281

70 μl of kit dilution buffer+30 μL of cell lysate, or 30 μl of lysis buffer for the blanks, are added to each well of the kit plate. Incubation is carried out for 2 h with gentle agitation at ambient temperature.

The wells are rinsed 4 times with 400 μl of kit washing buffer. Incubation is carried out with 100 μl of anti-phospho MET antibody for 1 hour at ambient temperature.

The wells are rinsed 4 times with 400 μl of kit washing buffer. Incubation is carried out with 100 μl of anti-rabbit HRP antibody for 30 minutes at ambient temperature (except for the wells of chromogen alone).

The wells are rinsed 4 times with 400 μl of kit washing buffer. 100 μL of chromogen are introduced and incubation is carried out for 30 minutes in the dark at ambient temperature.

The reaction is stopped with 100 μl of stop solution. The plate is read without delay at 450 nM, 0.1 second on a Wallac Victor plate reader.

### C) Test C: Measurement of Cell Proliferation by ¹⁴C-Thymidine Pulse

The cells are seeded into Cytostar 96-well plates in 180 μl for 4 hours at 37° C. and 5% CO₂: HCT116 cells in a proportion of 2500 cells per well in DMEM medium+10% foetal calf serum+1% L-glutamine, and MKN45 cells in a proportion of 7500 cells per well in RPMI medium+10% foetal calf serum+1% L-glutamine. After these 4 hours of incubation, the products are added in 10 μl as a 20-fold concentrated solution according to the dilution method mentioned for the ELISA. The products are tested at 10 concentrations in duplicate from 10 000 nM to 0.3 nM with an increment of 3.

After treatment for 72 h, 10 μl of ¹⁴C-thymidine at 10 μCi/ml are added so as to obtain 0.1 μCi per well. The ¹⁴C-thymidine incorporation is measured on a Micro-Beta machine (Perkin-Elmer) after 24 hours of pulse and 96 h of treatment.

All the steps of the assay are automated on BIOMEK 2000 or TECAN stations.

The results obtained with this test B for the products of formula (I) as examples in the experimental section are such that the IC50 is less than 10 microM, and in particular less than 1 microM.

The results obtained for the products as examples in the experimental section are given in the pharmacological results table hereinafter, as follows:

for test A, the sign + corresponds to less than 500 nM and the sign ++ corresponds to less than 100 nM;
for test B, the sign + corresponds to greater than 500 nM and the sign ++ corresponds to less than 100 nM;
for test C, the sign + corresponds to less than 10 microM and the sign ++ corresponds to less than 1 microM.

### Pharmacological Results Table:

| Example | Test A | Test B | Test C |
|---|---|---|---|
| 1 | + | | |
| 2 | ++ | + | ++ |
| 3 | ++ | ++ | ++ |
| 4 | ++ | ++ | ++ |
| 5 | ++ | ++ | ++ |
| 6 | ++ | | ++ |
| 7 | ++ | ++ | ++ |
| 8 | ++ | ++ | ++ |
| 9 | ++ | + | ++ |
| 10 | ++ | ++ | ++ |
| 11 | ++ | ++ | ++ |
| 12 | ++ | ++ | ++ |
| 13 | ++ | ++ | ++ |
| 14 | ++ | ++ | ++ |
| 15 | ++ | ++ | ++ |
| 16 | ++ | ++ | ++ |

1) A product of formula (I): 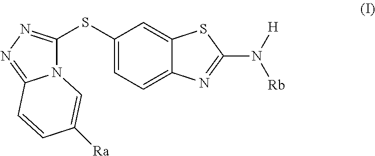
in which:
Ra represents a hydrogen atom; a halogen atom; an aryl radical; or a heteroaryl radical, these aryl and heteroaryl radicals being optionally substituted as indicated hereinafter;
Rb represents a hydrogen atom, an Rc, —COORc or —CO-Rc radical or a —CO—NRcRd radical;
where Rc represents an alkyl, cycloalkyl, heterocycloalkyl, aryl or heteroaryl radical, all these radicals being optionally substituted as indicated hereinafter;
Rd represents a hydrogen atom or an alkyl or cycloalkyl radical;
all the alkyl, cycloalkyl, heterocycloalkyl, aryl and heteroaryl radicals defined above being optionally substituted with one or more radicals chosen from halogen atoms, and hydroxyl, alkoxy, CN, CF₃, —NR1R2, heterocycloalkyl, —COOH, —COOalk, —CONR1R2 and —NR1COR2 radicals;
the alkyl and cycloalkyl radicals also being optionally substituted with an aryl or heteroaryl radical, themselves optionally substituted with one or more radicals chosen from halogen atoms and hydroxyl, alkyl, alkoxy and NR3R4 radicals;
the cycloalkyl, heterocycloalkyl, aryl or heteroaryl radicals also being optionally substituted with an alkyl radical, itself optionally substituted with one or more radicals chosen from halogen atoms and hydroxyl, O-heterocycloalkyl, alkyl, alkoxy and NR3R4 radicals;
NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
the cyclic radicals that R1 and R2 or R3 and R4, respectively, can form with the nitrogen atom to which they are attached, being optionally substituted with one or more radicals, which may be identical or different, chosen from halogen atoms, hydroxyl, oxo, alkoxy, NH₂, NHalk and N(alk)₂ radicals, and alkyl, phenyl, CH₂-phenyl and heteroaryl radicals, such that, in the latter radicals, the alkyl, phenyl and heteroaryl radicals are themselves optionally substituted with one or more radicals chosen from halogen atoms and the following radicals: hydroxyl, alkyl and alkoxy containing from 1 to 4 carbon atoms, NH₂, NHalk and N(alk)₂;
all the alkyl (alk) and alkoxy radicals above containing from 1 to 6 carbon atoms, racemic, enantiomeric and diastereoisomeric isomers, and pharmaceutically acceptable salts thereof.

2) The product of claim 1, in which:
Ra represents a hydrogen atom; a halogen atom; or an aryl or heteroaryl radical, these aryl and heteroaryl radicals being optionally substituted as indicated hereinafter;
Rb represents a hydrogen atom, a —CO—Rc radical or a —CO—NRcRd radical;
where Rc represents an alkyl radical or a cycloalkyl radical, both optionally substituted with one or more radicals chosen from hydroxyl, alkoxy, NR1R2, heterocycloalkyl, aryl and heteroaryl radicals, themselves optionally substituted as indicated hereinafter;
Rd represents a hydrogen atom or an alkyl radical;
all the alkyl, cycloalkyl, heterocycloalkyl, aryl and heteroaryl radicals defined above being optionally substituted with one or more radicals chosen from halogen atoms, and hydroxyl, alkoxy, heterocycloalkyl, —NR1R2, —COOH, —COOalk and —CONR1R2 radicals;
the aryl or heteroaryl radicals also being optionally substituted with an alkyl radical, itself optionally substituted with one or more radicals chosen from halogen atoms and hydroxyl, O-heterocycloalkyl and alkoxy radicals;
NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different, chosen from halogen atoms, hydroxyl and alkoxy radicals, and alkyl, phenyl and CH₂-phenyl radicals, in which the alkyl or phenyl radicals are themselves optionally substituted with one or more radicals, which may be identical or different, chosen from halogen atoms and alkyl, hydroxyl, alkoxy, NH₂, NHalk and N(alk)₂ radicals;
all the alkyl (alk) or alkoxy radicals above containing from 1 to 6 carbon atoms, racemic, enantiomeric and diastereoisomeric isomers, and pharmaceutically acceptable salts thereof.

3) The product according to claim 1, in which:
Ra represents a hydrogen atom; a halogen atom; a phenyl radical optionally substituted as indicated hereinafter; or a pyrazolyl radical optionally substituted with a heterocycloalkyl radical or with an alkyl radical, itself optionally substituted with a hydroxyl radical or with an O-heterocycloalkylradical;
Rb represents a hydrogen atom, a —CO—Rc radical or a —CO—NRcRd radical;
where Rc represents an alkyl or cycloalkyl radical, both optionally substituted with one or more radicals chosen from the radicals hydroxyl, alkoxy, NR1R2 and phenyl, itself optionally substituted with one or more radicals chosen from halogen atoms, and hydroxyl, alkoxy, alkyl, NH₂, NHalk and N(alk)₂ radicals;
Rd represents a hydrogen atom or an alkyl radical;
NR1R2 is such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, or phenyl radicals, themselves optionally substituted; or R1 and R2 form, with the nitrogen atom to which they are attached, a cyclic radical containing from 4 to 7 ring members and optionally another heteroatom chosen from O, S, N and NH, this radical, including the possible NH that it contains, being optionally substituted;
NR3R4 being such that: either R3 and R4, which may be identical or different, represent a hydrogen atom or an alkyl radical optionally substituted with one or more radicals, which may be identical or different, chosen from hydroxyl or alkoxy radicals; or R3 and R4 form, with the nitrogen atom to which they are attached, a cyclic radical containing from 4 to 7 ring members and optionally another heteroatom chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different;
all the alkyl (alk) or alkoxy radicals above containing from 1 to 4 carbon atoms, racemic, enantiomeric and diastereoisomeric isomers, and pharmaceutically acceptable salts thereof.

4) The product according to claim 1, in which:
Ra represents a hydrogen atom; a halogen atom; or a phenyl radical optionally substituted with one or more radicals chosen from halogen atoms and alkyl radicals; or a pyrazolyl radical optionally substituted with a piperidyl radical or with an alkyl radical, itself optionally substituted with a hydroxyl radical or with a tetrahydro-2H-pyran-2-yloxy radical;
Rb represents a hydrogen atom, a —CO-Rc radical or a —CO—NRcRd radical;
where Rc represents an alkyl or cycloalkyl radical optionally substituted with one or more radicals chosen from hydroxyl, alkoxy and NR1R2 radicals;
Rd represents a hydrogen atom;
NR1R2 being such that: either R1 and R2, which may be identical or different, represent a hydrogen atom or an alkyl radical optionally substituted with one or more radicals, which may be identical or different, chosen from hydroxyl, alkoxy, NH₂, NHalk and N(alk)₂ radicals; or R1 and R2 form, with the nitrogen atom to which they are attached, a cyclic radical containing from 4 to 7 ring members and optionally another heteroatom chosen from O, S, N and NH, optionally substituted with an alkyl, phenyl or —CH₂-phenyl radical, the latter radicals being themselves optionally substituted with one or more radicals, which may be identical or different, chosen from halogen atoms and alkyl, hydroxyl, alkoxy, NH₂, NHalk and N(alk)₂ radicals;
all the alkyl (alk) or alkoxy radicals above containing from 1 to 4 carbon atoms,
racemic, enantiomeric and diastereoisomeric isomers, and pharmaceutically acceptable salts thereof.

5) The product according to claim 1, in which:
Ra represents a hydrogen atom; an iodine atom; a phenyl radical optionally substituted with one or two radicals chosen from halogen atoms and a methyl radical; or a pyrazolyl radical optionally substituted with a piperidyl radical or with an ethyl radical, itself optionally substituted with a hydroxyl radical or with a tetrahydro-2H-pyran-2-yloxy radical;
Rb represents a hydrogen atom, a CO-Rc radical or a —CO—NRcRd radical;
where Rc represents a cyclopropyl radical or an alkyl radical optionally substituted with an alkoxy or NR1R2 radical;
Rd represents a hydrogen atom;
NR1R2 being such that: either R1 and R2, which may be identical or different, represent a hydrogen atom or an alkyl radical; or R1 and R2 form, with the nitrogen atom to which they are attached, a morpholinyl or piperazinyl radical optionally substituted on the second nitrogen atom with an alkyl radical;
the alkyl and alkoxy radicals above containing from 1 to 4 carbon atoms,
racemic, enantiomeric and diastereoisomeric isomers, and pharmaceutically acceptable salts thereof.

6) The product according to claim 1, corresponding to the following formulae:
N-[6-([1,2,4]triazolo[4,3-a]pyridin-3-ylsulphanyl)-1,3-benzothiazol-2-yl]cyclopropanecarboxamide
1-[2-(morpholin-4-yl)ethyl]-3-[6-([1,2,4]triazolo[4,3-a]pyridin-3-ylsulphanyl)-1,3-benzothiazol-2-yl]urea
1-[2-(4-methylpiperazin-1-yl)ethyl]-3-[6-([1,2,4]triazolo[4,3-a]pyridin-3-ylsulphanyl)-1,3-benzothiazol-2-yl]urea
1-(2-methoxyethyl)-3-[6-([1,2,4]triazolo[4,3-a]pyridin-3-ylsulphanyl)-1,3-benzothiazol-2-yl]urea
6-[(6-iodo[1,2,4]triazolo[4,3-a]pyridin-3-yl)sulphanyl]-1,3-benzothiazol-2-amine
6-{[6-(4-fluorophenyl)[1,2,4]triazolo[4,3-a]pyridin-3-yl]sulphanyl}-1,3-benzothiazol-2-amine
N-{6-[6-(4-fluorophenyl)[1,2,4]triazolo[4,3-a]pyridin-3-ylsulphanyl]-1,3-benzothiazol-2-yl}cyclopropanecarboxamide
6-{[6-(1-methyl-1H-pyrazol-4-yl)[1,2,4]triazolo[4,3-a]pyridin-3-yl]sulphanyl}-1,3-benzothiazol-2-amine
N-(6-{[6-(1-methyl-1H-pyrazol-4-yl)[1,2,4]triazolo[4,3-a]pyridin-3-yl]sulphanyl}-1,3-benzothiazol-2-yl)cyclopropanecarboxamide
N-(6-{[6-(1H-pyrazol-4-yl)[1,2,4]triazolo[4,3-a]pyridin-3-yl]sulphanyl}-1,3-benzothiazol-2-yl)cyclopropanecarboxamide
N-(6-{[6-((3-fluoro-4-methyl)phenyl)[1,2,4]triazolo[4,3-a]pyridin-3-yl]sulphanyl}-1,3-benzothiazol-2-yl)cyclopropanecarboxamide
N-(6-{[6-(3-fluorophenyl)[1,2,4]triazolo[4,3-a]pyridin-3-yl]sulphanyl}-1,3-benzothiazol-2-yl)cyclopropanecarboxamide
N-(6-{[6-(1-[2-(tetrahydro-2H-pyran-2-yloxy)ethyl]-1H-pyrazol-4-yl)[1,2,4]triazolo[4,3-a]pyridin-3-yl]sulphanyl}-1,3-benzothiazol-2-yl)cyclopropanecarboxamide
N-(6-{[6-(1-(2-hydroxyethyl)-1H-pyrazol-4-yl)[1,2,4]triazolo[4,3-a]pyridin-3-yl]sulphanyl}-1,3-benzothiazol-2-yl)cyclopropanecarboxamide
N-(6-{[6-(1-piperidin-4-yl-1H-pyrazol-4-yl) [1,2,4]triazolo[4,3-a]pyridin-3-yl]sulphanyl}-1,3-benzothiazol-2-yl)cyclopropanecarboxamide
and pharmaceutically acceptable salts thereof.

7) A process for preparing the product according to claim 1, according to scheme 1 as defined hereinafter: 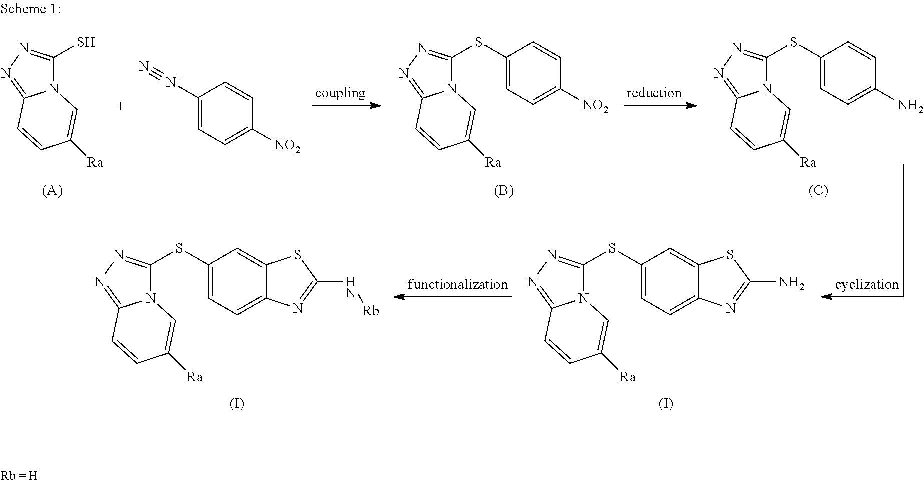
in which the substituents Ra and Rb have the meanings indicated in claim 1.

8) A process for preparing the product according to claim 1, according to scheme 2 as defined hereinafter: 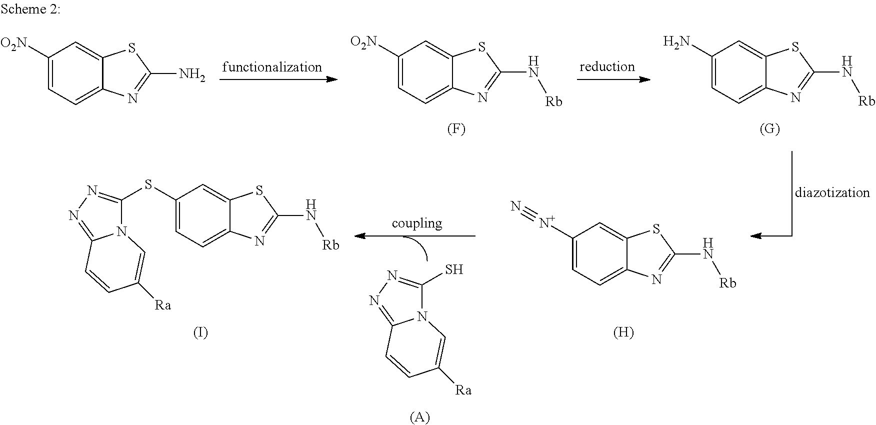
in which the substituents Ra and Rb have the meanings indicated in claim 1.

9) A process for preparing the product according to claim 1, according to scheme 3 as defined hereinafter: 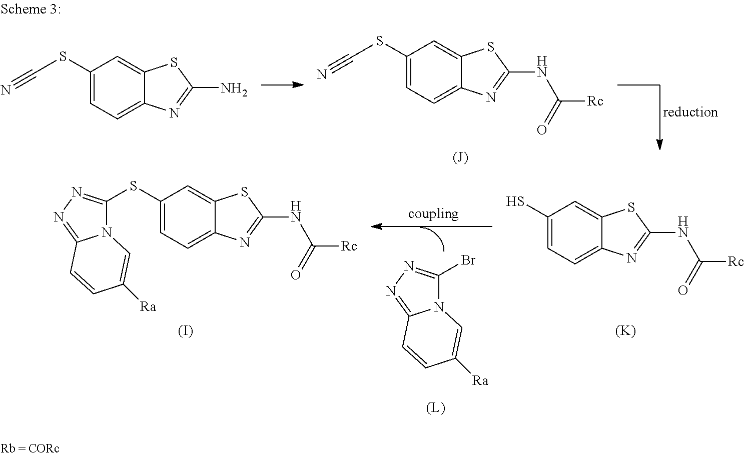
in which the substituents Ra and Rc have the meanings indicated in claim 1.

10) A pharmaceutical composition comprising the product of claim 1, and pharmaceutically acceptable salts thereof.

11) A pharmaceutical composition comprising the product of claim 6, and pharmaceutically acceptable salts thereof.

12) A pharmaceutical composition containing, as active ingredient, at least one product according to claim 1, or a pharmaceutically acceptable salt of said product or a prodrug of said product, and a pharmaceutically acceptable carrier.

13) A method of inhibiting the activity of the MET protein kinase and mutant forms thereof in a patient in need thereof comprising administering to said patient a therapeutically effective amount of the product according to claim 1, or pharmaceutically acceptable salts thereof.

14) The method of claim 13, wherein said protein kinase is in a cell culture.

15) A method of treating or preventing a disease in a patient in need thereof comprising administering to said patient a therapeutically effective amount of the product according to claim 1, wherein said disease is chosen from the following groups: blood vessel proliferation disorders, fibrotic disorders, “mesangial” cell proliferation disorders, metabolic disorders, allergies, asthma, thrombosis, nervous system diseases, retinopathy, psoriasis, rheumatoic arthritis, diabetes, muscle degeneration and cancers.

16) A method of treating cancers in a patient in need thereof comprising administering to said patient a therapeutically effective amount of the product according to claim 1.

17) The method according to claim 16, wherein solid or liquid tumours are treated.

18) The method according to claim 16, for wherein said cancers are resistant to cytotoxic agents.

19) The method according to claim 16, wherein primary tumours and/or metastases are treated, in particular in gastric, hepatic, renal, ovarian, colon, prostate and lung (NSCLC and SCLC) cancers, glioblastomas, thyroid, bladder or breast cancers, in melanoma, in lymphoid or myeloid hematopoietic tumours, in sarcomas, in brain, larynx or lympathic system tumours, bone cancers and pancreatic cancers.

20) (canceled)

21) (canceled)

22) The product according to claim 1, wherein said product is a kinase inhibitor.

23) The product according to claim 1, wherein said product is a MET inhibitor.

24) A product having one of the following formulas: 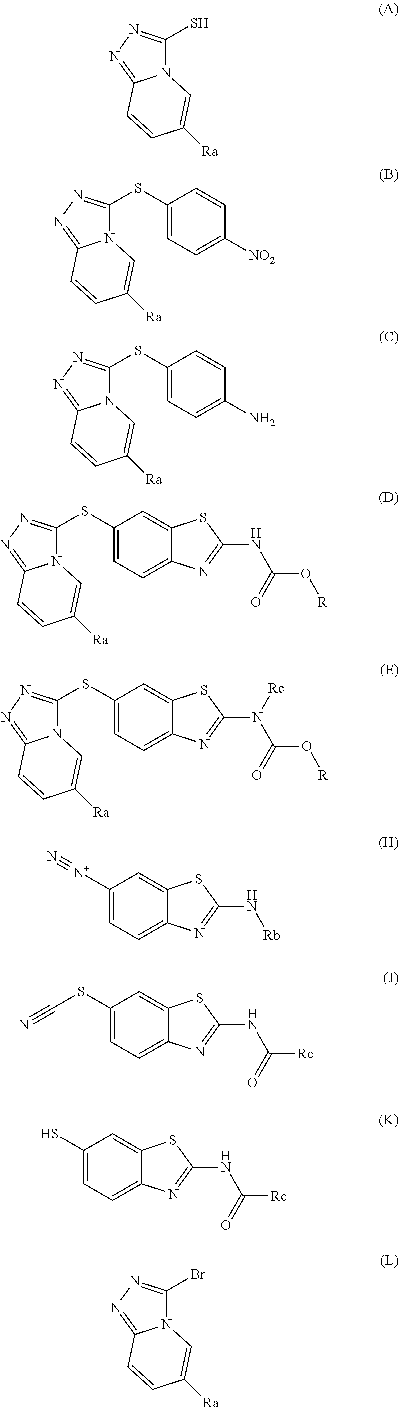 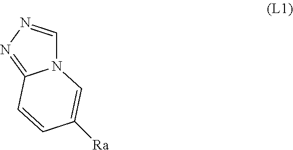
in which Ra, Rb and Rc have the definitions indicated in claim 1, and R represents a t-butyl or phenyl radical.
